(19) United States
(12) Patent Application Publication
(10) Pub. No.: US 2023/0248741 A1
(43) Pub. Date: Aug. 10, 2023

(54) Compositions and Methods for Lowering Triglycerides in a Subject with Reduced Kidney Function and Diabetes Mellitus

(71) Applicant: Amarin Pharmaceuticals Ireland Limited, Dublin (IE)

(72) Inventor: Sephy Philip, Bedminster, NJ (US)

(21) Appl. No.: 17/949,046

(22) Filed: Sep. 20, 2022

Related U.S. Application Data
(63) Continuation of application No. 16/292,732, filed on Mar. 5, 2019.
(60) Provisional application No. 62/639,093, filed on Mar. 6, 2018.

(51) Int. Cl. A61K 31/557; A61P 9/00

(52) U.S. Cl. CPC A61K 31/557; A61P 9/00

(57) ABSTRACT

In various embodiments, the present disclosure provides compositions and methods for treating and/or preventing cardiovascular-related diseases in subject in need thereof having triglyceride levels of about 200 mg/dL to about 499 mg/dL, diabetes mellitus, and reduced kidney function.

### PRIORITY CLAIM

This application is a continuation of U.S. application Ser. No. 16/292,732, filed Mar. 5, 2019, which claims priority to U.S. Provisional Application No. 62/639,093, filed on Mar. 6, 2018, the entire contents of which is incorporated herein by reference and relied upon.

### BACKGROUND

Cardiovascular disease is one of the leading causes of death in the United States and most European countries. It is estimated that over 70 million people in the United States alone suffer from a cardiovascular disease or disorder, including but not limited to high blood pressure, coronary heart disease, dyslipidemia, congestive heart failure and stroke.

### SUMMARY

In some aspects, the present disclosure provides methods of treating or preventing cardiovascular-related disease in a subject having baseline fasting triglycerides of about 200 mg/dl to about 500 mg/dl, reduced kidney function, and diabetes mellitus, the method comprising administering to the subject a composition comprising about 4 g of at least 96% ethyl eicosapentaenoate (E-EPA), by weight, of total fatty acids present in the composition per day for 12 weeks, wherein after administration of the composition, the subject experiences a reduction in triglycerides and cardiovascular risk factors without raising low density lipoprotein cholesterol (LDL-C) levels compared to a placebo control.

In some embodiments, the subject has an estimated glomerular filtration rate (GFR) levels less than 90 mL/min/1.73 m² for 3 months or greater. In another embodiment, reduced kidney function is measured by the subject's GFR or the subject's estimated GFR.

In some embodiments, the risk factors are hs-CRP; Lp-PLA2; ox-LDL; Apo C III; Apo B; RLP-C; vLDL-TG; HDL-C; vLDL-C; TC; and non-HDL-C.

In another embodiment, the subject has at least one cardiovascular-related disease. In yet another embodiment, the subject has coronary heart disease or a coronary heart disease risk equivalent.

In some embodiments, the subject has one or more of: (a) a fasting baseline high-sensitivity C-reactive protein (hsCRP) level of at least about 2 mg/L, (b) a fasting baseline lipoprotein-associated phospholipase A2 (Lp-PLA₂) level of 170 ng/dl; (c) a fasting oxidized low density lipoprotein (ox-LDL) level of at least about 55 mg/dl; (d) a fasting baseline apolipoprotein C-III (Apo C-III) level of at least about 40 mg/dl; (e) a fasting baseline apolipoprotein B (Apo B) level of at least about 100 mg/dl; (f) a fasting baseline level of remnant-like particles cholesterol (RLP-C) level of at least about 40 mg/dl; (g) a fasting baseline level of very low density lipoprotein triglyceride (vLDL-TG) level of at least about 180 mg/dl; (h) a fasting baseline high density lipoprotein cholesterol (HDL-C) level of not more than about 40 mg/dl; (i) a fasting baseline very low density lipoprotein (vLDL-C) level of at least about 40 mg/dl; (j) a fasting baseline total cholesterol (TC) level of greater than about 160 mg/dl; (k) a baseline fasting non-high density lipoprotein cholesterol (non-HDL-C) level of at least about 120 mg/dl; (l) a fasting baseline albumin level at least about 4.5 mg/dl; (m) a fasting baseline serum creatine level of at least about 0.9 mg/dl; or (n) a fasting baseline blood urea nitrogen (BUN) level of at least about 18 mg/dl.

In some embodiments, the subject exhibits a reduction in one or more of: hsCRP; Lp-PLA2; ox-LDL; Apo C III; Apo B; RLP-C; vLDL-TG; HDL-C; vLDL-C; TC; and non-HDL-C following administration of E-EPA as compared to placebo control and/or baseline. In yet another embodiment, the subject exhibits one or more of: (a) a reduction in a hsCRP level of at least about 25%, (b) a reduction in a Lp-PLA₂ level of at least about 15%; (c) a reduction in an ox-LDL level of at least about 15%; (d) a reduction in an Apo C-III level of at least about 20%; (e) a reduction in an Apo B level of at least about 10%; (f) a reduction in a RLP-C level of at least about 35%; (g) a reduction in a vLDL-TG level of at least about 20%; (h) a reduction in a HDL-C level of at least about 7%; (i) a reduction in a vLDL-C level of at least about 15%; (j) a reduction in a TC level of at least about 10%; and (k) a reduction in a non-HDL-C level of at least about 10% as compared to baseline.

In one embodiment, the subject has an LDL-C level of about 40 mg/dl to about 100 mg/dl prior to said administering. In another embodiment, the subject exhibits a reduction in LDL-C levels following administration of E-EPA as compared to placebo control and/or baseline. In one embodiment, the subject does not experience a statistically significant change in serum creatinine, eGFR levels, BUN and/or albumin levels following administration of E-EPA as compared to placebo control and/or baseline. In yet another embodiment, the subject exhibits an increase in plasma and/or red blood cell (RBC) levels of E-EPA following administration of E-EPA as compared to placebo control and/or baseline.

In some embodiments, the subject is on concomitant statin therapy and/or on stable statin therapy.

These and other embodiments of the present disclosure will be disclosed in further detail herein below.

### BRIEF DESCRIPTION OF THE DRAWINGS

FIG. 1 shows percentages of subjects having an eGFR of less than about 60 mL/min/1.73 m² for ≥3 months and at least about 90 mL/min/1.73 m² for ≥3 months. The percentages of subjects are stratified by those having a baseline fasting triglyceride (TG) level of at least about 150 mg/dL, or those having a baseline fasting TG level of less than about 150 mg/dL and a baseline high-density lipoprotein cholesterol (HDL-C) level of more than about 40 mg/dL.

FIG. 2 shows the median percent change in lipid/lipoprotein, apolipoprotein, and inflammatory parameters in patients having reduced kidney function with eGFR of at least about 90 mL/min/1.73 m² for ≥3 months and diabetes mellitus at baseline to week 12 who have been administered E-EPA 4 g/day or placebo. * P<0.0001; † P<0.001; ‡ P<0.01; § P<0.05 vs placebo.

FIG. 3 shows the median percent change in lipid/lipoprotein, apolipoprotein, and inflammatory parameters in patients having reduced kidney function with eGFR of at least about 90 mL/min/1.73 m² for ≥3 months and hsCRP levels ≥2.0 mg/L at baseline to week 12 who have been administered E-EPA 4 g/day or placebo. * P<0.0001; † P<0.001; ‡ P<0.01; § P<0.05; NS, not significant vs placebo.

FIG. 4 shows the median percent change in lipid/lipoprotein, apolipoprotein, and inflammatory parameters in patients having chronic kidney disease at baseline to week 12 who have been administered E-EPA 4 g/day or placebo.

### DETAILED DESCRIPTION

While the present disclosure is capable of being embodied in various forms, the description below of several embodiments is made with the understanding that the present disclosure is to be considered as an exemplification of the disclosure and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

In one embodiment, the disclosure provides a method for treatment and/or prevention of cardiovascular-related diseases. The term “cardiovascular-related disease” herein refers to any disease or disorder of the heart or blood vessels (i.e. arteries and veins) or any symptom thereof. Non-limiting examples of cardiovascular-related disease and disorders include hypertriglyceridemia, hypercholesterolemia, mixed dyslipidemia, coronary heart disease, vascular disease, stroke, atherosclerosis, arrhythmia, hypertension, myocardial infarction, and other cardiovascular events.

The term “treatment” in relation to a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing, or treating symptoms of the disease or disorder. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

Abbreviations: TGs, triglycerides; LDL-C, low-density lipoprotein cholesterol; non-HDL-C, non-high-density lipoprotein cholesterol; TC, total cholesterol; HDL-C, high density lipoprotein cholesterol; VLDL-C, very-low-density lipoprotein cholesterol; VLDL-TG, very-low-density lipoprotein triglycerides; RLP-C, remnant-like particle cholesterol; Apo, apolipoprotein; Ox-LDL, oxidized low-density lipoprotein; Lp-PLA₂, lipoprotein-associated phospholipase A₂; hsCRP, high-sensitivity C-reactive protein; eGFR, estimated glomerular filtration rate.

In one embodiment, the present disclosure provides a method of blood lipid therapy comprising administering to a subject or subject group in need thereof a pharmaceutical composition as described herein. In another embodiment, the subject or subject group has hypertriglyceridemia, hypercholesterolemia, mixed dyslipidemia, and/or very high triglycerides.

In another embodiment, the subject or subject group being treated has a baseline triglyceride level (or mean or median baseline triglyceride level in the case of a subject group), fed or fasting, of about 200 mg/dl to about 500 mg/dl. In another embodiment, the subject or subject group has a baseline LDL-C level (or mean or median baseline LDL-C level), despite stable statin therapy, of about 40 mg/dl to about 115 or about 40 to about 100 mg/dl.

In one embodiment, the subject or subject group being treated in accordance with methods of the disclosure is on concomitant statin therapy, for example atorvastatin, rosuvastatin, or simvastatin therapy (with or without ezetimibe). In another embodiment, the subject is on concomitant stable statin therapy at time of initiation of ultra-pure EPA therapy.

In another embodiment, the subject or subject group being treated in accordance with methods of the disclosure has a body mass index (BMI or mean BMI) of not more than about 45 kg/m².

In one embodiment, the disclosure provides a method of lowering triglycerides in a subject on stable statin therapy having baseline fasting triglycerides of about 200 mg/dl to about 500 mg/dl, the method comprising administering to the subject a pharmaceutical composition comprising about 1 g to about 4 g of EPA (e.g. ultra-pure EPA), wherein upon administering the composition to the subject daily for a period of about 12 weeks the subject exhibits at least 10%, at least 15%, at least 20%, at least 25%, at least 30%, at least 35%, at least 40%, at least 45%, at least 50%, at least 55%, at least 60%, at least 65%, at least 70%, or at least 75% lower fasting triglycerides than a control subject maintained on stable statin therapy (and optionally placebo matching the ultra-pure EPA) without concomitant ultra-pure EPA for a period of about 12 weeks, wherein the control subject also has baseline fasting triglycerides of about 200 mg/dl to about 500 mg/dl. The term “stable statin therapy” herein means that the subject, subject group, control subject, or control subject group in question has been taking a stable daily dose of a statin (e.g. atorvastatin, rosuvastatin or simvastatin) for at least 4 weeks prior to the baseline fasting triglyceride measurement (the “qualifying period”). For example, a subject or control subject on stable statin therapy would receive a constant daily (i.e. the same dose each day) statin dose for at least 4 weeks immediately prior to baseline fasting triglyceride measurement. In one embodiment, the subject's and control subject's LDL-C is maintained between about 40 mg/dl and about 115 mg/dl or about 40 mg/dl to about 100 mg/dl during the qualifying period. The subject and control subject are then continued on their stable statin dose for the 12 week period post baseline.

In one embodiment, the statin is administered to the subject and the control subject in an amount of about 1 mg to about 500 mg, about 5 mg to about 200 mg, or about 10 mg to about 100 mg, for example about 1 mg, about 2 mg, about 3 mg, about 4 mg, about 5 mg, about 6 mg, about 7 mg, about 8 mg, about 9 mg, or about 10 mg; about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg. In another embodiment, the subject (and optionally the control subject) has a baseline LDL-C level, despite stable statin therapy, of about 40 mg/dl to about 115 mg/dl or about 40 mg/dl to about 100 mg/dl. In another embodiment, the subject and/or control subject has a body mass index (BMI or mean BMI) of not more than about 45 kg/m².

In another embodiment, the disclosure provides a method of lowering triglycerides in a subject group on stable statin therapy having mean baseline fasting triglycerides of about 200 mg/dl to about 500 mg/dl, the method comprising administering to members of the subject group a pharmaceutical composition comprising about 1 g to about 4 g of ultra-pure EPA per day, wherein upon administering the composition to the members of the subject group daily for a period of about 12 weeks the subject group exhibits at least 10%, at least 15%, at least 20%, at least 25%, at least 30%, at least 35%, at least 40%, at least 45%, at least 50%, at least 55%, at least 60%, at least 65%, at least 70%, or at least 75% lower mean fasting triglycerides than a control subject group maintained on stable statin therapy without concomitant ultra-pure EPA (optionally with matching placebo) for a period of about 12 weeks, wherein the control subject group also has mean baseline fasting triglycerides of about 200 mg/dl to about 500 mg/dl. In a related embodiment, the stable statin therapy will be sufficient such that the subject group has a mean LDL-C level at least about 40 mg/dl and not more than about 100 mg/dl or about 40 mg/dl to about 100 mg/dl for the 4 weeks immediately prior to the baseline fasting triglyceride measurement.

In another embodiment, the disclosure provides a method of lowering triglycerides in a subject group on stable statin therapy and having a mean baseline fasting triglyceride level of about 200 mg/dl to about 500 mg/dl, the method comprising administering to members of the subject group a pharmaceutical composition comprising about 1 g to about 4 g of ultra-pure EPA, wherein upon administering the composition to members of the subject group daily for a period of about 12 weeks the subject group exhibits: (a) at least 10%, at least 15%, at least 20%, at least 25%, at least 30%, at least 35%, at least 40%, at least 45%, at least 50%, at least 55%, at least 60%, at least 65%, at least 70%, or at least 75% lower mean fasting triglycerides by comparison with a control subject group maintained on stable statin therapy without concomitant ultra-pure EPA (optionally with matching placebo) for a period of about 12 weeks, and (b) no serum LDL-C increase, no statistically significant serum LDL-C increase, a serum LDL-C decrease, or the subject is statistically non-inferior to the control subjects (statin plus optional placebo) in regard to serum LDL-C elevation) no increase in mean serum LDL-C levels compared to baseline, wherein the control subject also has mean baseline fasting triglycerides of about 200 mg/dl to about 500 mg/dl.

In another embodiment, the disclosure provides a method of lowering triglycerides in a subject on stable statin therapy and having a mean baseline fasting triglyceride level of about 200 mg/dl to about 500 mg/dl, the method comprising administering to the subject a pharmaceutical composition comprising about 1 g to about 4 g of ultra-pure EPA, wherein upon administering the composition to the subject daily for a period of about 12 weeks the subject exhibits (a) at least 10%, at least 15%, at least 20%, at least 25%, at least 30%, at least 35%, at least 40%, at least 45%, at least 50%, at least 55%, at least 60%, at least 65%, at least 70%, or at least 75% lower fasting triglycerides by comparison with a control subject maintained on stable statin therapy without concomitant ultra-pure EPA for a period of about 12 weeks and (b) no increase in serum LDL-C levels compared to baseline, wherein the control subject also has baseline fasting triglycerides of about 200 mg/dl to about 500 mg/dl.

In another embodiment, the disclosure provides a method of lowering triglycerides in a subject group on stable statin therapy and having a mean baseline fasting triglyceride level of about 200 mg/dl to about 500 mg/dl, the method comprising administering to members of the subject group a pharmaceutical composition comprising about 1 g to about 4 g of ultra-pure EPA, wherein upon administering the composition to the members of the subject group daily for a period of about 12 weeks the subject group exhibits: (a) at least 10%, at least 15%, at least 20%, at least 25%, at least 30%, at least 35%, at least 40%, at least 45%, at least 50%, at least 55%, at least 60%, at least 65%, at least 70%, or at least 75% lower mean fasting triglycerides and (b) at least 5%, at least 10%, at least 15%, at least 20%, at least 25%, at least 30%, at least 35%, at least 40%, at least 45%, or at least 50% lower mean serum LDL-C levels by comparison with a control subject group maintained on stable statin therapy without concomitant ultra-pure EPA (optionally with matching placebo) for a period of about 12 weeks, no serum LDL-C increase, no statistically significant serum LDL-C increase, no statistically significant serum LDL-C increase, a serum LDL-C decrease, or the subject group is statistically non-inferior to the control subject group (statin plus optional placebo) in regard to serum LDL-C elevation), wherein the control subject group also has mean baseline fasting triglycerides of about 200 mg/dl to about 500 mg/dl.

In another embodiment, the disclosure provides a method of lowering triglycerides in a subject group on stable statin therapy and having a mean baseline fasting triglyceride level of about 200 mg/dl to about 500 mg/dl, the method comprising administering to members of the subject group a pharmaceutical composition comprising about 1 g to about 4 g of ultra-pure EPA, wherein upon administering the composition to the members of the subject group daily for a period of about 12 weeks the subject group exhibits (a) at least 10%, at least 15%, at least 20%, at least 25%, at least 30%, at least 35%, at least 40%, at least 45%, at least 50%, at least 55%, at least 60%, at least 65%, at least 70%, or at least 75% lower mean fasting triglycerides and (b) at least 5%, at least 10%, at least 15%, at least 20%, at least 25%, at least 30%, at least 35%, at least 40%, at least 45%, or at least 50% lower mean serum LDL-C levels by comparison with a control subject group maintained on stable statin therapy without concomitant ultra-pure EPA (optionally with matching placebo) for a period of about 12 weeks, no serum LDL-C increase, no statistically significant serum LDL-C increase, no statistically significant serum LDL-C increase, a serum LDL-C decrease, or the subject group is statistically non-inferior to the control subject group (statin plus optional placebo) in regard to serum LDL-C elevation), wherein the control subject group also has mean baseline fasting triglycerides of about 200 mg/dl to about 500 mg/dl.

In some embodiments, the disclosure provides methods of treating or preventing cardiovascular-related disease in a subject having baseline fasting triglycerides of about 200 mg/dl to about 500 mg/dl, reduced kidney function, and diabetes mellitus, the method comprising administering to the subject a composition comprising about 4 g of E-EPA, by weight, of total fatty acids present in the composition per day for 12 weeks, wherein after administration of the composition, the subject experiences a reduction in triglycerides and cardiovascular risk factors without raising low density lipoprotein cholesterol (LDL-C) levels compared to a placebo control. In some embodiments, the cardiovascular risk factors are hsCRP; Lp-PLA₂; ox-LDL; Apo C III; Apo B; RLP-C; vLDL-TG; HDL-C; vLDL-C; TC; and non-HDL-C.

In another embodiment, the subject or subject group being treated in accordance with methods of the disclosure exhibits a fasting baseline absolute plasma level of free total fatty acid (or mean thereof) not greater than about 300 nmol/ml, not greater than about 250 nmol/ml, not greater than about 200 nmol/ml, not greater than about 150 nmol/ml, not greater than about 100 nmol/ml, or not greater than about 50 nmol/ml.

In another embodiment, the subject or subject group being treated in accordance with methods of the disclosure exhibits a fasting baseline absolute plasma level of free EPA (or mean thereof in the case of a subject group) not greater than about 0.70 nmol/ml, not greater than about 0.65 nmol/ml, not greater than about 0.60 nmol/ml, not greater than about 0.55 nmol/ml, not greater than about 0.50 nmol/ml, not greater than about 0.45 nmol/ml, or not greater than about 0.40 nmol/ml. In another embodiment, the subject or subject group being treated in accordance with methods of the disclosure exhibits a baseline fasting plasma level (or mean thereof) of free EPA, expressed as a percentage of total free fatty acid, of not more than about 3%, not more than about 2.5%, not more than about 2%, not more than about 1.5%, not more than about 1%, not more than about 0.75%, not more than about 0.5%, not more than about 0.25%, not more than about 0.2%, or not more than about 0.15%. In one such embodiment, free plasma EPA and/or total fatty acid levels are determined prior to initiating therapy.

In another embodiment, the subject or subject group being treated in accordance with methods of the disclosure exhibits a fasting baseline absolute plasma level of free EPA (or mean thereof) not greater than about 1 nmol/ml, not greater than about 0.75 nmol/ml, not greater than about 0.50 nmol/ml, not greater than about 0.4 nmol/ml, not greater than about 0.35 nmol/ml, or not greater than about 0.30 nmol/ml.

In another embodiment, the subject or subject group being treated in accordance with methods of the disclosure exhibits a fasting baseline plasma, serum, or red blood cell membrane EPA level not greater than about 150 □g/ml, not greater than about 125 □g/ml, not greater than about 100 □g/ml, not greater than about 95 □g/ml, not greater than about 75 □g/ml, not greater than about 60 □g/ml, not greater than about 50 □g/ml, not greater than about 40 □g/ml, not greater than about 30 □g/ml, or not greater than about 25 □g/ml.

In another embodiment, the subject or subject group being treated in accordance with methods of the disclosure exhibits a fasting baseline plasma, serum, or red blood cell EPA level not greater than about 150 □g/ml, not greater than about 125 □g/ml, not greater than about 100 □g/ml, not greater than about 95 □g/ml, not greater than about 75 □g/ml, not greater than about 60 □g/ml, not greater than about 50 □g/ml, not greater than about 40 □g/ml, not greater than about 30 □g/ml, not greater than about 25 □g/ml, not greater than about 20 □g/ml, not greater than about 15 □g/ml, or not greater than about 10 □g/ml and has reduced kidney function (e.g., eGFR<90 mL/min/1.73 m² for ≥3 months), diabetes mellitus, and a fasting baseline plasma, serum, or red blood cell EPA level of about 25 □g/ml, about 20 □g/ml, about 15 □g/ml, or about 10 □g/ml. In another embodiment, the subject or subject group being treated in accordance with the methods of the present disclosure has reduced kidney function (e.g., eGFR<90 mL/min/1.73 m² for ≥3 months), diabetes mellitus, and has a fasting baseline plasma level EPA of about 25 □g/ml and/or a fasting baseline red blood cell EPA level of about 10 □g/ml.

In another embodiment, the subject or subject group being treated in accordance with methods of the disclosure exhibits a fasting baseline total cholesterol (TC) level of greater than about 50 mg/dl, of greater than about 60 mg/dl, greater than about 70 mg/dl, greater than about 80 mg/dl, greater than about 90 mg/dl, greater than about 100 mg/dl, greater than about 110 mg/dl, greater than about 120 mg/dl, greater than about 125 mg/dl, greater than about 130 mg/dL, greater than about 140 mg/dl, greater than about 150 mg/dl, greater than about 160 mg/dl, greater than about 170 mg/dl, or greater than about 200 mg/dl.

In another embodiment, the subject or subject group being treated in accordance with the methods of the present disclosure has reduced kidney function (e.g., eGFR<90 mL/min/1.73 m² for ≥3 months), diabetes mellitus, and a fasting baseline TC level of greater than about 50 mg/dl, of greater than about 60 mg/dl, greater than about 70 mg/dl, greater than about 80 mg/dl, greater than about 90 mg/dl, greater than about 100 mg/dl, greater than about 110 mg/dl, greater than about 120 mg/dl, greater than about 125 mg/dl, greater than about 130 mg/dL, greater than about 140 mg/dl, greater than about 150 mg/dl, greater than about 160 mg/dl, greater than about 170 mg/dl, or greater than about 200 mg/dl.

In some embodiments, the subject or subject group being treated in accordance with methods of the disclosure exhibits a fasting baseline non-HDL-C level of about 100 mg/dl to about 400 mg/dl, for example at least about 100 mg/dl, at least about 110 mg/dl, at least about 120 mg/dl, at least about 130 mg/dl, at least about 140 mg/dl, at least about 150 mg/dl, at least about 160 mg/dl, at least about 170 mg/dl, at least about 180 mg/dl, at least about 190 mg/dl, at least about 200 mg/dl, at least about 210 mg/dl, at least about 220 mg/dl, at least about 230 mg/dl, at least about 240 mg/dl, at least about 250 mg/dl, at least about 260 mg/dl, at least about 270 mg/dl, at least about 280 mg/dl, at least about 290 mg/dl, or at least about 300 mg/dl.

In another embodiment, the subject or subject group being treated in accordance with the methods of the present disclosure has reduced kidney function (e.g., eGFR<90 mL/min/1.73 m² for ≥3 months), diabetes mellitus, and has a fasting baseline non-HDL-C level of about 100 mg/dl to about 400 mg/dl, for example at least about 100 mg/dl, at least about 110 mg/dl, at least about 120 mg/dl, at least about 130 mg/dl, at least about 140 mg/dl, at least about 150 mg/dl, at least about 160 mg/dl, at least about 170 mg/dl, at least about 180 mg/dl, at least about 190 mg/dl, at least about 200 mg/dl, at least about 210 mg/dl, at least about 220 mg/dl, at least about 230 mg/dl, at least about 240 mg/dl, at least about 250 mg/dl, at least about 260 mg/dl, at least about 270 mg/dl, at least about 280 mg/dl, at least about 290 mg/dl, or at least about 300 mg/dl.

In some embodiments, the subject or subject group being treated in accordance with methods of the disclosure exhibits a fasting baseline vLDL-C level of about 30 mg/dl to about 200 mg/dl, for example at least about 30 mg/dl, at least about 40 mg/dl, at least about 50 mg/dl, at least about 60 mg/dl, at least about 70 mg/dl, at least about 80 mg/dl, at least about 90 mg/dl, at least about 100 mg/dl, at least about 110 mg/dl, at least about 120 mg/dl, at least about 130 mg/dl, at least about 140 mg/dl, at least about 150 mg/dl, at least about 160 mg/dl.

In another embodiment, the subject or subject group being treated in accordance with the methods of the present disclosure has reduced kidney function (e.g., eGFR<90 mL/min/1.73 m² for ≥3 months), diabetes mellitus, and a fasting baseline vLDL-C level of about 30 mg/dl to about 200 mg/dl, for example at least about 30 mg/dl, at least about 40 mg/dl, at least about 50 mg/dl, at least about 60 mg/dl, at least about 70 mg/dl, at least about 80 mg/dl, at least about 90 mg/dl, at least about 100 mg/dl, at least about 110 mg/dl, at least about 120 mg/dl, at least about 130 mg/dl, at least about 140 mg/dl, at least about 150 mg/dl, at least about 160 mg/dl.

In some embodiments, the subject or subject group being treated in accordance with methods of the disclosure exhibits a fasting baseline HDL-C level of about 10 to about 100 mg/dl, for example not more than about 90 mg/dl, not more than about 80 mg/dl, not more than about 70 mg/dl, not more than about 60 mg/dl, not more than about 60 mg/dl, not more than about 50 mg/dl, not more than about 40 mg/dl, not more than about 35 mg/dl, not more than about 30 mg/dl, not more than about 25 mg/dl, not more than about 20 mg/dl, or not more than about 15 mg/dl.

In another embodiment, the subject or subject group being treated in accordance with the methods of the present disclosure has reduced kidney function (e.g., eGFR<90 mL/min/1.73 m² for ≥3 months), diabetes mellitus, and a fasting baseline HDL-C level of about 10 to about 100 mg/dl, for example not more than about 90 mg/dl, not more than about 80 mg/dl, not more than about 70 mg/dl, not more than about 60 mg/dl, not more than about 60 mg/dl, not more than about 50 mg/dl, not more than about 40 mg/dl, not more than about 35 mg/dl, not more than about 30 mg/dl, not more than about 25 mg/dl, not more than about 20 mg/dl, or not more than about 15 mg/dl.

In some embodiments, the subject or subject group being treated in accordance with methods of the disclosure exhibits a fasting baseline vLDL-TG level of about 30 mg/dl to about 300 mg/dl, for example at least about 30 mg/dl, at least about 40 mg/dl, at least about 50 mg/dl, at least about 60 mg/dl, at least about 70 mg/dl, at least about 80 mg/dl, at least about 90 mg/dl, at least about 100 mg/dl, at least about 110 mg/dl, at least about 120 mg/dl, at least about 130 mg/dl, at least about 140 mg/dl, at least about 150 mg/dl, at least about 160 mg/dl, at least about 170 mg/dl, at least about 180 mg/dl, at least about 190 mg/dl, at least about 200 mg/dl, at least about 210 mg/dl, at least about 220 mg/dl, at least about 230 mg/dl, at least about 240 mg/dl, at least about 250 mg/dl, at least about 260 mg/dl, at least about 270 mg/dl, at least about 280 mg/dl, at least about 290 mg/dl, or at least about 300 mg/dl.

In another embodiment, the subject or subject group being treated in accordance with the methods of the present disclosure has reduced kidney function (e.g., eGFR<90 mL/min/1.73 m² for ≥3 months), diabetes mellitus, and a fasting baseline vLDL-TG level of about 30 mg/dl to about 300 mg/dl, for example at least about 30 mg/dl, at least about 40 mg/dl, at least about 50 mg/dl, at least about 60 mg/dl, at least about 70 mg/dl, at least about 80 mg/dl, at least about 90 mg/dl, at least about 100 mg/dl, at least about 110 mg/dl, at least about 120 mg/dl, at least about 130 mg/dl, at least about 140 mg/dl, at least about 150 mg/dl, at least about 160 mg/dl, at least about 170 mg/dl, at least about 180 mg/dl, at least about 190 mg/dl, at least about 200 mg/dl, at least about 210 mg/dl, at least about 220 mg/dl, at least about 230 mg/dl, at least about 240 mg/dl, at least about 250 mg/dl, at least about 260 mg/dl, at least about 270 mg/dl, at least about 280 mg/dl, at least about 290 mg/dl, or at least about 300 mg/dl.

In some embodiments, the subject or subject group being treated in accordance with methods of the disclosure exhibits a fasting baseline RLP-C level of at least about 3 mg/dl, at least about 4 mg/dl, at least about 5 mg/dl, at least about 6 mg/dl, at least about 7 mg/dl, at least about 8 mg/dl, at least about 9 mg/dl, at least about 10 mg/dl, at least about 11 mg/dl, at least about 12 mg/dl, at least about 13 mg/dl, at least about 14 mg/dl, at least about 15 mg/dl, at least about 16 mg/dl, at least about 17 mg/dl, at least about 18 mg/dl, at least about 19 mg/dl, or at least about 20 mg/dl.

In another embodiment, the subject or subject group being treated in accordance with the methods of the present disclosure has reduced kidney function (e.g., eGFR<90 mL/min/1.73 m² for ≥3 months), diabetes mellitus, and a fasting baseline RLP-C level of at least about 3 mg/dl, at least about 4 mg/dl, at least about 5 mg/dl, at least about 6 mg/dl, at least about 7 mg/dl, at least about 8 mg/dl, at least about 9 mg/dl, at least about 10 mg/dl, at least about 11 mg/dl, at least about 12 mg/dl, at least about 13 mg/dl, at least about 14 mg/dl, at least about 15 mg/dl, at least about 16 mg/dl, at least about 17 mg/dl, at least about 18 mg/dl, at least about 19 mg/dl, or at least about 20 mg/dl.

In some embodiments, the subject or subject group being treated in accordance with methods of the disclosure exhibits a fasting baseline Apo B level at least about 50 mg/dl, at least about 100 mg/dl, at least about 150 mg/dl, or at least about 200 mg/dl.

In another embodiment, the subject or subject group being treated in accordance with the methods of the present disclosure has reduced kidney function (e.g., eGFR<90 mL/min/1.73 m² for ≥3 months), diabetes mellitus, and a fasting baseline Apo B level at least about 50 mg/dl, at least about 100 mg/dl, at least about 150 mg/dl, or at least about 200 mg/dl.

In some embodiments, the subject or subject group being treated in accordance with methods of the disclosure exhibits a fasting baseline Apo C III level between about 30 mg/dl to about 125 mg/dl. For example, at least about 30 mg/dl, at least about 40 mg/dl, at least about 50 mg/dl, at least about 55 mg/dl, at least about 60 mg/dl, at least about 65 mg/dl, at least about 70 mg/dl, at least about 80 mg/dl, at least about 85 mg/dl, at least about 90 mg/dl, at least about 95 mg/dl, at least about 100 mg/dl, at least about 105 mg/dl, at least about 110 mg/dl, at least about 115 mg/dl, or at least about 120 mg/dl.

In another embodiment, the subject or subject group being treated in accordance with the methods of the present disclosure has reduced kidney function (e.g., eGFR<90 mL/min/1.73 m² for ≥3 months), diabetes mellitus, and a fasting baseline Apo C III level between about 30 mg/dl to about 125 mg/dl. For example, at least about 30 mg/dl, at least about 40 mg/dl, at least about 50 mg/dl, at least about 55 mg/dl, at least about 60 mg/dl, at least about 65 mg/dl, at least about 70 mg/dl, at least about 80 mg/dl, at least about 85 mg/dl, at least about 90 mg/dl, at least about 95 mg/dl, at least about 100 mg/dl, at least about 105 mg/dl, at least about 110 mg/dl, at least about 115 mg/dl, or at least about 120 mg/dl.

In some embodiments, the subject or subject group being treated in accordance with methods of the disclosure exhibits a fasting baseline oxidized LDL level between about 30 mg/dl to about 60 mg/dl. For example, at least about 30 mg/dl, at least about 35 mg/dl, at least about 40 mg/dl, at least about 45 mg/dl, at least about 50 mg/dl, or at least about 55 mg/dl.

In another embodiment, the subject or subject group being treated in accordance with the methods of the present disclosure has reduced kidney function (e.g., eGFR<90 mL/min/1.73 m² for ≥3 months), diabetes mellitus, and a fasting baseline ox-LDL level between about 30 mg/dl to about 60 mg/dl. For example, at least about 30 mg/dl, at least about 35 mg/dl, at least about 40 mg/dl, at least about 45 mg/dl, at least about 50 mg/dl, or at least about 55 mg/dl.

In some embodiments, the subject or subject group being treated in accordance with methods of the disclosure exhibits a fasting baseline lipoprotein associated Lp-PLA₂ level of about 30 mg/dl to about 300 mg/dl, for example at least about 30 mg/dl, at least about 40 mg/dl, at least about 50 mg/dl, at least about 60 mg/dl, at least about 70 mg/dl, at least about 80 mg/dl, at least about 90 mg/dl, at least about 100 mg/dl, at least about 110 mg/dl, at least about 120 mg/dl, at least about 130 mg/dl, at least about 140 mg/dl, at least about 150 mg/dl, at least about 160 mg/dl, at least about 170 mg/dl, at least about 180 mg/dl, at least about 190 mg/dl, at least about 200 mg/dl, at least about 210 mg/dl, at least about 220 mg/dl, at least about 230 mg/dl, at least about 240 mg/dl, at least about 250 mg/dl, at least about 260 mg/dl, at least about 270 mg/dl, at least about 280 mg/dl, at least about 290 mg/dl, or at least about 300 mg/dl.

In another embodiment, the subject or subject group being treated in accordance with the methods of the present disclosure has reduced kidney function (e.g., eGFR<90 mL/min/1.73 m² for ≥3 months), diabetes mellitus, and a fasting baseline Lp-PLA₂ level of about 30 ng/ml to about 300 ng/ml, for example at least about 30 ng/ml, at least about 40 ng/ml, at least about 50 ng/ml, at least about 60 ng/ml, at least about 70 ng/ml, at least about 80 ng/ml, at least about 90 ng/ml, at least about 100 ng/ml, at least about 110 ng/ml, at least about 120 ng/ml, at least about 130 ng/ml, at least about 140 ng/ml, at least about 150 ng/ml, at least about 160 ng/ml, at least about 170 ng/ml, at least about 180 ng/ml, at least about 190 ng/ml, at least about 200 ng/ml, at least about 210 ng/ml, at least about 220 ng/ml, at least about 230 ng/ml, at least about 240 ng/ml, at least about 250 ng/ml, at least about 260 ng/ml, at least about 270 ng/ml, at least about 280 ng/ml, at least about 290 ng/ml, or at least about 300 ng/ml.

In some embodiments, the subject or subject group being treated in accordance with methods of the disclosure exhibits a fasting baseline hsCRP level between about 2 mg/L to about 10 mg/L, about 3 mg/L to about 15 mg/L, about 5 mg/L to about 30 mg/L, about 4 mg/L to about 20 mg/L, about 10 mg/L to about 25 mg/L, about 6 mg/L to about 15 mg/L, about 4 mg/L to about 25 mg/L, or about 2 mg/L to about 30 mg/L.

In another embodiment, the subject or subject group being treated in accordance with the methods of the present disclosure has reduced kidney function (e.g., eGFR<90 mL/min/1.73 m² for ≥3 months), diabetes mellitus, and a fasting baseline hsCRP level between about 2 mg/L to about 10 mg/L, about 3 mg/L to about 15 mg/L, about 5 mg/L to about 30 mg/L, about 4 mg/L to about 20 mg/L, about 10 mg/L to about 25 mg/L, about 6 mg/L to about 15 mg/L, about 4 mg/L to about 25 mg/L, or about 2 mg/L to about 30 mg/L.

In some embodiments, the subject or subject group being treated in accordance with methods of the disclosure exhibits a fasting baseline BUN level between about 2 mg/L to about 10 mg/L, about 3 mg/L to about 15 mg/L, about 5 mg/L to about 30 mg/L, about 4 mg/L to about 20 mg/L, about 10 mg/L to about 25 mg/L, about 6 mg/L to about 15 mg/L, about 4 mg/L to about 25 mg/L, or about 2 mg/L to about 30 mg/L.

In another embodiment, the subject or subject group being treated in accordance with the methods of the present disclosure has reduced kidney function (e.g., eGFR<90 mL/min/1.73 m² for ≥3 months), diabetes mellitus, and a fasting baseline BUN level between about 2 mg/L to about 10 mg/L, about 3 mg/L to about 15 mg/L, about 5 mg/L to about 30 mg/L, about 4 mg/L to about 20 mg/L, about 10 mg/L to about 25 mg/L, about 6 mg/L to about 15 mg/L, about 4 mg/L to about 25 mg/L, or about 2 mg/L to about 30 mg/L.

In some embodiments, the subject or subject group being treated in accordance with methods of the disclosure exhibits a fasting baseline serum creatinine level between about 0.6 mg/dl to about 1.3 mg/dl, about 0.3 mg/dl to about 2.0 mg/dl, or to about 0.7 mg/dl to about 1.5 mg/dl. For example, at least about 0.6 mg/dl, at least about 0.7 mg/dl, at least about 0.8 mg/dl, at least about 0.9 mg/dl, at least about 1 mg/dl, at least about 1.1 mg/dl, at least about 1.2 mg/dl, at least about 1.3 mg/dl, at least about 1.4 mg/dl, at least about 1.5 mg/dl, at least about 1.6 mg/dl, at least about 1.7 mg/dl, at least about 1.8 mg/dl, or at least about 1.9 mg/dl.

In another embodiment, the subject or subject group being treated in accordance with the methods of the present disclosure has reduced kidney function (e.g., eGFR<90 mL/min/1.73 m² for ≥3 months), diabetes mellitus, and a fasting baseline serum creatine level between about 0.6 mg/dl to about 1.3 mg/dl, about 0.3 mg/dl to about 2.0 mg/dl, or to about 0.7 mg/dl to about 1.5 mg/dl. For example, at least about 0.6 mg/dl, at least about 0.7 mg/dl, at least about 0.8 mg/dl, at least about 0.9 mg/dl, at least about 1 mg/dl, at least about 1.1 mg/dl, at least about 1.2 mg/dl, at least about 1.3 mg/dl, at least about 1.4 mg/dl, at least about 1.5 mg/dl, at least about 1.6 mg/dl, at least about 1.7 mg/dl, at least about 1.8 mg/dl, or at least about 1.9 mg/dl.

In some embodiments, the subject or subject group being treated in accordance with methods of the disclosure exhibits a fasting baseline albumin level of about 3.0 g/dl to about 6 g/dl, about 3.5 g/dl to about 5.5 g/dl, or about 4 g/dl to about 5 g/dl. For example, at least about 3.0 g/dl, at least about 3.5 g/dl, at least about 4.0 g/dl, at least about 4.5 g/dl, at least about 5.0 g/dl, or at least about 5.5 g/dl.

In another embodiment, the subject or subject group being treated in accordance with the methods of the present disclosure has reduced kidney function (e.g., eGFR<90 mL/min/1.73 m² for ≥3 months), diabetes mellitus, and a fasting baseline albumin level of about 3.0 g/dl to about 6 g/dl, about 3.5 g/dl to about 5.5 g/dl, or about 4 g/dl to about 5 g/dl. For example, at least about 3.0 g/dl, at least about 3.5 g/dl, at least about 4.0 g/dl, at least about 4.5 g/dl, at least about 5.0 g/dl, or at least about 5.5 g/dl.

In another embodiment, methods of the present disclosure comprise a step of measuring the subject's (or subject group's mean) baseline lipid profile prior to initiating therapy. In another embodiment, methods of the disclosure comprise the step of identifying a subject or subject group having one or more of the following: baseline non-HDL-C value (or mean) of about 200 mg/dl to about 400 mg/dl, for example at least about 210 mg/dl, at least about 220 mg/dl, at least about 230 mg/dl, at least about 240 mg/dl, at least about 250 mg/dl, at least about 260 mg/dl, at least about 270 mg/dl, at least about 280 mg/dl, at least about 290 mg/dl, or at least about 300 mg/dl; baseline total cholesterol value (or mean) of about 250 mg/dl to about 400 mg/dl, for example at least about 260 mg/dl, at least about 270 mg/dl, at least about 280 mg/dl or at least about 290 mg/dl; baseline vLDL-C value (or mean) of about 140 mg/dl to about 200 mg/dl, for example at least about 150 mg/dl, at least about 160 mg/dl, at least about 170 mg/dl, at least about 180 mg/dl, or at least about 190 mg/dl; baseline HDL-C value (or mean) of about 10 to about 100 mg/dl, for example not more than about 90 mg/dl, not more than about 80 mg/dl, not more than about 70 mg/dl, not more than about 60 mg/dl, not more than about 60 mg/dl, not more than about 50 mg/dl, not more than about 40 mg/dl, not more than about 35 mg/dl, not more than about 30 mg/dl, not more than about 25 mg/dl, not more than about 20 mg/dl, or not more than about 15 mg/dl; and/or baseline LDL-C value (or mean) of about 30 to about 300 mg/dl, for example not less than about 40 mg/dl, not less than about 50 mg/dl, not less than about 60 mg/dl, not less than about 70 mg/dl, not less than about 90 mg/dl, or not less than about 90 mg/dl.

In another embodiment, methods of the present disclosure comprise increasing or otherwise improving the subject's (or subject group's mean) renal function, such as long term renal function, as measured by the subject's (or subject group's mean) eGFR value. In some embodiments, methods of the present disclosure comprise determining the subject's or the subject group's mean baseline eGFR level prior to initiating therapy. In another embodiment, methods of the disclosure comprise the step of identifying a subject or subject group having one or more of the following: baseline fasting triglyceride value (or mean) of at least about 150 mg/dL or less than about 150 mg/dL; baseline non-fasting triglyceride value (or mean) of at least about 150 mg/dL or less than about 150 mg/dL; baseline HDL-C value (or mean) of about 10 to about 100 mg/dl, for example not more than about 90 mg/dl not, not more than about 80 mg/dl, not more than about 70 mg/dl, not more than about 60 mg/dl, not more than about 60 mg/dl, not more than about 50 mg/dl, not more than about 40 mg/dl, not more than about 35 mg/dl, not more than about 30 mg/dl, not more than about 25 mg/dl, not more than about 20 mg/dl, or not more than about 15 mg/dl; and/or baseline LDL-C value (or mean) of about 30 to about 300 mg/dl, for example not less than about 40 mg/dl, not less than about 50 mg/dl, not less than about 60 mg/dl, not less than about 70 mg/dl, not less than about 90 mg/dl, or not less than about 90 mg/dl. In some embodiments, the subject or subjects are at least about forty-five (45) years of age, have diabetes and/or a CV disease, such as an atherosclerotic CV disease, and are on statin therapy, such as stable or concomitant statin therapy, or have otherwise been treated with a statin. In some embodiments, the subject or subjects do not have end stage renal disease and are not receiving hemodialysis or peritoneal dialysis.

In another embodiment, the present disclosure provides methods of reducing cardiovascular risk in patients having reduced kidney function (e.g., estimated glomerular filtration rate (eGFR)<90 mL/min/1.73 m² for ≥3 months) and diabetes mellitus. In some embodiments, the present disclosure provides methods of reducing cardiovascular risk in patients having reduced kidney function (e.g., eGFR<90 mL/min/1.73 m² for ≥3 months) and high-sensitivity C-Reactive protein (hsCRP) levels ≥2.0 mg/L; In some embodiments, the present disclosure provides method of reducing cardiovascular risk in patients having chronic kidney disease (e.g., eGFR<60 mL/min/1.73 m² for ≥3 months). In some embodiments, the present disclosure provides a method of reducing cardiovascular risk in patients having fasting baseline triglycerides of about 200 mg/dl to about 500 mg/dl, LDL-C control, and LDL-C levels of about 40 mg/dl to 100 mg/dl, the method comprising administering to the subject a pharmaceutical composition comprising 4 g/day E-EPA, wherein upon administering the composition to the subject for a period of 12 weeks, the subject exhibits a reduction in triglycerides without raising LDL-C levels and significantly improved atherogenic and inflammatory parameters compared to baseline or placebo control. In some embodiments, the atherogenic and inflammatory parameters include non-HDL-C, TC, VLDL-C, Lp-PLA2, apolipoprotein B, apolipoprotein-C III, total cholesterol, HDL-C, RLP-C or VLDL-TG. In some embodiments, the subject exhibits substantially no change in creatinine, eGFR and/or albumin. In some embodiments, the subject exhibits an increase in EPA and/or plasma levels in red blood cells (RBCs) compared to baseline or placebo control.

In a related embodiment, upon treatment in accordance with the present disclosure, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks, or about 1 week, the subject or subject group exhibits one or more of the following outcomes:

(a) a reduction in triglyceride levels compared to baseline or placebo control (e.g. a subject on stable statin plus placebo matching the EPA treatment group);

(b) substantially no change (e.g., increase), no statistically significant change, or an increase in non-HDL-C levels compared to baseline or placebo control;

(c) a reduction in non-HDL-C levels compared to baseline or placebo control;

(d) substantially no change (e.g. increase), no statistically significant change, or an increase in HDL-C levels;

(e) substantially no change, no statistically significant change, or an increase in LDL-C levels compared to baseline or placebo control;

(f) a reduction in LDL-C levels compared to baseline or placebo control;

(g) a reduction in Apo B levels compared to baseline or placebo control;

(h) a reduction in vLDL-C levels compared to baseline or placebo control;

(i) an increase in Apo A-I levels compared to baseline or placebo control;

(j) an increase in apo A-I/apo B ratio compared to baseline or placebo control;

(k) a reduction in lipoprotein (a) levels compared to baseline or placebo control;

(l) a reduction in mean LDL particle number compared to baseline or placebo control;

(m) an increase in mean LDL particle size compared to baseline or placebo control;

(n) a reduction in RLP-C compared to baseline or placebo control;

(o) a reduction in ox-LDL compared to baseline or placebo control;

(p) substantially no change, no statistically significant change, or a reduction in fasting plasma glucose (FPG) levels compared to baseline or placebo control;

(q) substantially no change, no statistically significant change, or a reduction in hemoglobin A_{1c} (HbA_{1c}) compared to baseline or placebo control;

(r) a reduction in homeostasis model insulin resistance compared to baseline or placebo control;

(s) a reduction in Lp-PLA₂ compared to baseline or placebo control;

(t) a reduction in intracellular adhesion molecule-1 compared to baseline or placebo control;

(u) a reduction in interleukin-6 compared to baseline or placebo control;

(v) a reduction in plasminogen activator inhibitor-1 compared to baseline or placebo control;

(w) a reduction in hsCRP compared to baseline or placebo control;

(x) an increase in serum, plasma and/or RBC EPA compared to baseline or placebo control;

(y) an increase in serum phospholipid and/or RBC membrane EPA compared to baseline or placebo control;

(z) a reduction in one or more of serum phospholipid and/or RBC content of docosahexaenoic acid (DHA), docosapentaenoic acid (DPA), arachidonic acid (AA), palmitic acid (PA), stearidonic acid (SA), or oleic acid (OA) compared to baseline or placebo control;

(aa) an increase in one or more of serum phospholipid and/or RBC content of DHA, DPA, AA, PA, SA, or OA compared to baseline or placebo control;

(bb) a reduction in total cholesterol levels compared to baseline or placebo control;

(cc) an increase or improvement in long-term renal function compared to baseline, placebo control, and/or a cohort (e.g., a propensity matched comparator cohort);

(dd) a reduction in vLDL-TG levels compared to baseline or placebo control;

(ee) a reduction in Apo C-III levels compared to baseline or placebo control;

(ff) substantially no change, no statistically significant change, or a reduction in serum creatine levels compared to baseline or placebo control;

(gg) substantially no change, no statistically significant change, or an increase in eGFR levels compared to baseline or placebo control;

(hh) substantially no change, no statistically significant change, or an increase in albumin levels compared to baseline or placebo control; and/or

(ii) substantially no change, no statistically significant change levels, or a reduction in blood urea nitrogen (BUN) levels;

In one embodiment, methods of the present disclosure comprise measuring baseline levels of one or more markers set forth in (a)-(ii) above prior to dosing the subject or subject group. In another embodiment, the methods comprise administering a composition as disclosed herein to the subject after baseline levels of one or more markers set forth in (a)-(ii) are determined, and subsequently taking an additional measurement of said one or more markers.

In another embodiment, upon treatment with a composition of the present disclosure, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks, or about 1 week, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, any 22 or more of, any 23 or more, any 24 or more, or all 25 of outcomes (a)-(ii) described immediately above.

In another embodiment, upon treatment with a composition of the present disclosure, the subject or subject group exhibits one or more of the following outcomes:

(a) a reduction in triglyceride level of at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) as compared to baseline or placebo control (e.g. a subject on statin and placebo matching the EPA treatment group);

(b) substantially no change, no statistically significant change, or an increase in non-HDL-C levels of less than 30% increase, less than 20% increase, less than 10% increase, less than 5% increase, or no increase in non-HDL-C levels as compared to baseline or placebo control;

(c) a reduction in non-HDL-C levels of at least about 1%, at least about 3%, at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) as compared to baseline or placebo control;

(d) substantially no change, no change, or an increase in HDL-C levels of at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) as compared to baseline or placebo control;

(e) substantially no change, no statistically significant change, or an increase in LDL-C levels by a less than 60% increase, less than 50% increase, less than 40% increase, less than 30% increase, less than 20% increase, less than 10% increase, less than 5% increase as compared to baseline or placebo control;

(f) a reduction in LDL-C levels of at least about 5%, at least about 10%, at least about 15%, at least about 20%, at least about 25%, at least about 30%, at least about 35%, at least about 40%, at least about 45%, at least about 50%, at least about 55%, at least about 55%, or at least about 75% (actual % change or median % change) as compared to baseline or placebo control;

(g) a reduction in Apo B levels of at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) as compared to baseline or placebo control;

(h) a reduction in vLDL-C levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(i) an increase in Apo A-I levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(j) an increase in apo A-I/Apo B ratio of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(k) a reduction in lipoprotein (a) levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(l) a reduction in mean LDL particle number of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(m) an increase in mean LDL particle size of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(n) a reduction in RLP-C of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(o) a reduction in ox-LDL of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(p) substantially no change, no statistically significant change, or a reduction in FPG of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(q) substantially no change, no statistically significant change, a reduction in HbA_{1c} of at least about 5%, at least about 10%, at least about 15%, at least about 20%, at least about 25%, at least about 30%, at least about 35%, at least about 40%, at least about 45%, or at least about 50% (actual % change or median % change) compared to baseline or placebo control;

(r) a reduction in homeostasis model index insulin resistance of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(s) a reduction in Lp-PLA₂ of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(t) a reduction in intracellular adhesion molecule-1 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(u) a reduction in interleukin-6 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(v) a reduction in plasminogen activator inhibitor-1 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(w) a reduction in high sensitivity C-reactive protein (hsCRP) of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(x) an increase in serum, plasma and/or RBC EPA of at least about 5%, at least about 10%, at least about 15%, at least about 20%, at least about 25%, at least about 30%, at least about 35%, at least about 40%, at least about 45%, at least about 50%, at least about 100%, at least about 200%, or at least about 400% (actual % change or median % change) compared to baseline or placebo control;

(y) an increase in serum phospholipid and/or RBC membrane EPA of at least about 5%, at least about 10%, at least about 15%, at least about 20%, at least about 25%, at least about 30%, at least about 35%, at least about 40%, at least about 45%, at least about 50%, at least about 100%, at least about 200%, or at least about 400% (actual % change or median % change) compared to baseline or placebo control;

(z) a reduction in one or more of serum phospholipid and/or red blood cell DHA, DPA, AA, SA, PA, and/or OA of at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) compared to baseline or placebo control;

(aa) an increase in one or more of serum phospholipid and/or red blood cell DHA, DPA, AA, SA, PA, and/or OA of at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) compared to baseline or placebo control

(bb) a reduction in total cholesterol of at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) compared to baseline or placebo control;

(cc) an increase or improvement in long-term renal function of at least about 0.5%, at least about 1%, at least about 2%, at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline, placebo, and/or cohort control;

(dd) a reduction in vLDL-TG of at least about 5%, at least about 10%, at least about 15%, at least about 20%, at least about 25%; or at least 30%, at least about 35%, at least about 40%, at least about 45%, at least about 50%, or at least about 100% (actual % change or median % change) compared to baseline or placebo control;

(ee) a reduction in Apo C III of at least about 5%, at least about 10%, at least about 15%, at least about 20%, or at least about 25%; at least about 30%, at least about 35%, at least about 40%, at least about 45%, at least about 50%, or at least about 100% (actual % change or median % change) compared to baseline or placebo control;

(ff) substantially no change, no statistically significant change, or a reduction in serum creatine of at least about 5%, at least about 10%, at least about 15%, at least about 20%, or at least about 25%; at least about 30%, at least about 35%, at least about 40%, at least about 45%, at least about 50%, or at least about 100% (actual % change or median % change) compared to baseline or placebo control;

(gg) substantially no change, no statistically significant change, or an increase in eGFR levels of at least about 5%, at least about 10%, at least about 15%, at least about 20%, or at least about 25%; at least about 30%, at least about 35%, at least about 40%, at least about 45%, at least about 50%, or at least about 100% (actual % change or median % change) compared to baseline or placebo control;

(hh) substantially no change, no statistically significant change, or an increase in albumin levels of at least about 5%, at least about 10%, at least about 15%, at least about 20%, or at least about 25%; at least about 30%, at least about 35%, at least about 40%, at least about 45%, at least about 50%, or at least about 100% (actual % change or median % change) compared to baseline or placebo control and/or

(ii) substantially no change, no statistically significant change, or a reduction in BUN levels of at least about 5%, at least about 10%, at least about 15%, at least about 20%, or at least about 25%; at least about 30%, at least about 35%, at least about 40%, at least about 45%, at least about 50%, or at least about 100% (actual % change or median % change) compared to baseline or placebo control.

In one embodiment, methods of the present disclosure comprise measuring baseline levels of one or more markers set forth in (a)-(ii) prior to dosing the subject or subject group. In another embodiment, the methods comprise administering a composition as disclosed herein to the subject after baseline levels of one or more markers set forth in (a)-(ii) are determined, and subsequently taking a second measurement of the one or more markers as measured at baseline for comparison thereto.

In another embodiment, upon treatment with a composition of the present disclosure, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks, or about 1 week, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, any 22 or more of, any 23 or more of, any 24 or more of, or all 27 or more of outcomes (a)-(ii) described immediately above.

Parameters (a)-(ii) can be measured in accordance with any clinically acceptable methodology. For example, triglycerides, total cholesterol, HDL-C and fasting blood sugar can be sampled from serum and analyzed using standard photometry techniques. VLDL-TG, LDL-C and VLDL-C can be calculated or determined using serum lipoprotein fractionation by preparative ultracentrifugation and subsequent quantitative analysis by refractometry or by analytic ultracentrifugal methodology. Apo A1, Apo B and hsCRP can be determined from serum using standard nephelometry techniques. Lipoprotein (a) can be determined from serum using standard turbidimetric immunoassay techniques. LDL particle number and particle size can be determined using nuclear magnetic resonance (NMR) spectrometry. Remnants lipoproteins and LDL-phospholipase A2 can be determined from EDTA plasma or serum and serum, respectively, using enzymatic immunoseparation techniques. Oxidized LDL, intercellular adhesion molecule-1 and interleukin-2 levels can be determined from serum using standard enzyme immunoassay techniques. These techniques are described in detail in standard textbooks, for example Tietz Fundamentals of Clinical Chemistry, 6^{th} Ed. (Burtis, Ashwood and Borter Eds.), WB Saunders Company.

In one embodiment, subjects fast for up to 12 hours prior to blood sample collection, for example about 10 hours.

In another embodiment, the subject being treated is in the highest risk category of Adult Treatment Panel (ATP) III Classification of LDL, Total, and HDL Cholesterol (mg/dL) (e.g. CHD or CHD Risk Equivalents (10-year risk >20%)). In another embodiment, the subject is in the ATP III Multiple (2+) risk factor category.

In one embodiment, the disclosure provides a method of lowering triglycerides in a subject in the highest risk category of Adult Treatment Panel (ATP) III Classification of LDL, Total, and HDL Cholesterol (mg/dL) (e.g. CHD or CHD Risk Equivalents (10-year risk >20%)). In another embodiment, the subject is in the ATP III Multiple (2+) risk factor category. In another embodiment, the method includes a step of identifying a subject in the ATP III Multiple (2+) risk factor category prior to administering ultra-pure E-EPA to the subject.

In another embodiment, the present disclosure provides a method of treating or preventing primary hypercholesterolemia and/or mixed dyslipidemia (Fredrickson Types IIa and IIb) in a patient in need thereof, comprising administering to the patient one or more compositions as disclosed herein. In a related embodiment, the present disclosure provides a method of reducing triglyceride levels in a subject or subjects when treatment with a statin or niacin extended-release monotherapy is considered inadequate (Frederickson type IV hyperlipidemia).

In another embodiment, the present disclosure provides a method of treating or preventing risk of recurrent nonfatal myocardial infarction in a patient with a history of myocardial infarction, comprising administering to the patient one or more compositions as disclosed herein.

In another embodiment, the present disclosure provides a method of slowing progression of or promoting regression of atherosclerotic disease in a patient in need thereof, comprising administering to a subject in need thereof one or more compositions as disclosed herein.

In another embodiment, the present disclosure provides a method of treating or preventing very high serum triglyceride levels (e.g. Types IV and V hyperlipidemia) in a patient in need thereof, comprising administering to the patient one or more compositions as disclosed herein.

In one embodiment, a composition of the disclosure is administered to a subject in an amount sufficient to provide a daily dose of EPA of about 1 mg to about 10,000 mg, about 25 mg to about 5000 mg, about 50 mg to about 3000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, or about 4100 mg.

In another embodiment, any of the methods disclosed herein are used in treatment of a subject or subjects that consume a traditional Western diet. In one embodiment, the methods of the disclosure include a step of identifying a subject as a Western diet consumer or a prudent diet consumer and then treating the subject if the subject is deemed a Western diet consumer. The term “Western diet” herein refers generally to a typical diet consisting of, by percentage of total calories, about 45% to about 50% carbohydrate, about 35% to about 40% fat, and about 10% to about 15% protein. A Western diet may alternately or additionally be characterized by relatively high intakes of red and processed meats, sweets, refined grains, and desserts, for example more than 50%, more than 60%, or more or 70% of total calories come from these sources.

In another embodiment, any of the methods disclosed herein are used in treatment of a subject or subjects that consume less than (actual or average) about 150 g, less than about 125 g, less than about 100 g, less than about 75 g, less than about 50 g, less than about 45 g, less than about 40 g, less than about 35 g, less than about 30 g, less than about 25 g, less than about 20 g, or less than about 15 g of fish per day.

In another embodiment, any of the methods disclosed herein are used in treatment of a subject or subjects that consume less than (actual or average) about 10 g, less than about 9 g, less than about 8 g, less than about 7 g, less than about 6 g, less than about 5 g, less than about 4 g, less than about 3 g, or less than about 2 g per day of omega-3 fatty acids from dietary sources.

In another embodiment, any of the methods disclosed herein are used in treatment of a subject or subjects that consume less than (actual or average) about 2.5 g, less than about 2 g, less than about 1.5 g, less than about 1 g, less than about 0.5 g, less than about 0.25 g, or less than about 0.2 g per day of EPA and DHA (combined) from dietary sources.

In one embodiment, compositions useful in various embodiments of the disclosure comprise a polyunsaturated fatty acid as an active ingredient. In another embodiment, such compositions comprise EPA as an active ingredient. The term “EPA” as used herein refers to eicosapentaenoic acid (e.g. eicosa-5,8,11,14,17-pentaenoic acid) and/or a pharmaceutically acceptable ester, derivative, conjugate, or salt thereof, or mixtures of any of the foregoing.

In one embodiment, the EPA comprises all-cis eicosa-5,8,11,14,17-pentaenoic acid. In another embodiment, the EPA is in the form of an eicosapentaenoic acid ester. In another embodiment, the EPA comprises a C₁-C₅ alkyl ester of EPA. In another embodiment, the EPA comprises eicosapentaenoic acid ethyl ester, eicosapentaenoic acid methyl ester, eicosapentaenoic acid propyl ester, or eicosapentaenoic acid butyl ester. In still another embodiment, the EPA comprises all-cis eicosa-5,8,11,14,17-pentaenoic acid ethyl ester.

In still other embodiments, the EPA comprises ethyl-EPA, lithium EPA, mono, di- or triglyceride EPA or any other ester or salt of EPA, or the free acid form of EPA. The EPA may also be in the form of a 2-substituted derivative or other derivative which slows down its rate of oxidation but does not otherwise change its biological action to any substantial degree.

The term “pharmaceutically acceptable” in the present context means that the substance in question does not produce unacceptable toxicity to the subject or interaction with other components of the composition.

In one embodiment, EPA present in a composition suitable for use according to the disclosure comprises ultra-pure EPA. The term “ultra-pure” as used herein with respect to EPA refers to a composition comprising at least 96% by weight EPA (as the term “EPA” is defined and exemplified herein). Ultra-pure EPA can comprise even higher purity EPA, for example at least 97% by weight EPA, at least 98% by weight EPA, or at least 99% by weight EPA, wherein the EPA is any form of EPA as set forth herein. Ultra-pure EPA can further be defined (e.g. impurity profile) by any of the description of EPA provided herein.

In some embodiments, EPA is present in a composition in an amount of about 50 mg to about 5000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, or about 2500 mg.

In various embodiments, one or more antioxidants can be present in the EPA (e.g. E-EPA or ultra pure E-EPA). Non-limiting examples of suitable antioxidants include tocopherol, lecithin, citric acid, and/or ascorbic acid. One or more antioxidants, if desired, are typically present in the EPA in an amount of about 0.01% to about 0.1%, by weight, or about 0.025% to about 0.05%, by weight.

In one embodiment, a composition of the disclosure contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of total fatty acids, docosahexaenoic acid or derivative thereof such as E-DHA, if any. In another embodiment, a composition of the disclosure contains substantially no docosahexaenoic acid or derivative thereof such as E-DHA. In still another embodiment, a composition of the disclosure contains no docosahexaenoic acid or E-DHA.

In another embodiment, EPA represents at least about 60%, at least about 70%, at least about 80%, at least about 90%, at least about 95%, at least about 97%, at least about 98%, at least about 99%, or 100%, by weight, of all fatty acids present in a composition useful in accordance with the disclosure.

In another embodiment, a composition of the disclosure contains less than 30%, less than 20%, less than 10%, less than 9%, less than 8%, less than 7%, less than 6%, less than 5%, less than 4%, less than 3%, less than 2%, less than 1%, less than 0.5%, or less than 0.25%, by weight of the total composition or by weight of the total fatty acid content, of any fatty acid other than EPA, or derivative thereof. Illustrative examples of a “fatty acid other than EPA” include linolenic acid (LA) or derivative thereof such as ethyl-linolenic acid, AA or derivative thereof such as ethyl-AA, DHA or derivative thereof such as ethyl-DHA, alpha-linolenic acid (ALA) or derivative thereof such as ethyl-ALA, stearadonic acid (STA) or derivative thereof such as ethyl-SA, eicosatrienoic acid (ETA) or derivative thereof such as ethyl-ETA, and/or DPA or derivative thereof such as ethyl-DPA.

In another embodiment, a composition of the disclosure has one or more of the following features: (a) eicosapentaenoic acid ethyl ester represents at least 96%, at least 97%, or at least 98%, by weight, of all fatty acids present in the composition; (b) the composition contains not more than 4%, not more than 3%, or not more than 2%, by weight, of total fatty acids other than eicosapentaenoic acid ethyl ester; (c) the composition contains not more than 0.6%, 0.5%, 0.4%, or 0.3% of any individual fatty acid other than eicosapentaenoic acid ethyl ester; (d) the composition has a refractive index (20° C.) of about 1 to about 2, about 1.2 to about 1.8, or about 1.4 to about 1.5; (e) the composition has a specific gravity (20° C.) of about 0.8 to about 1.0, about 0.85 to about 0.95, or about 0.9 to about 0.92; (f) the composition contains not more than 20 ppm, 15 ppm, or 10 ppm heavy metals, (g) the composition contains not more than 5 ppm, 4 ppm, 3 ppm, or 2 ppm arsenic, and/or (h) the composition has a peroxide value not more than 5, 4, 3, or 2 Meq/kg.

In another embodiment, a composition useful in accordance with the disclosure comprises, consists essentially of, or consists of at least 95% by weight ethyl eicosapentaenoate (E-EPA), about 0.2% to about 0.5% by weight ethyl octadecatetraenoate (ODTA-E), about 0.05% to about 0.25% by weight ethyl nonaecapentaenoate (NDPA-E), about 0.2% to about 0.45% by weight ethyl arachidonate (AA-E), about 0.3% to about 0.5% by weight ethyl eicosatetraenoate (ETA-E), and about 0.05% to about 0.32% ethyl heneicosapentaenoate (HPA-E). In another embodiment, the composition is present in a capsule shell. In still another embodiment, the capsule shell contains no chemically modified gelatin.

In another embodiment, compositions useful in accordance with the disclosure comprise, consist essentially of, or consist of at least 95%, 96%, or 97%, by weight, ethyl eicosapentaenoate, about 0.2% to about 0.5% by weight ODTA-E, about 0.05% to about 0.25% by weight NDPA-E, about 0.2% to about 0.45% by weight AA-E, about 0.3% to about 0.5% by weight ETA-E, and about 0.05% to about 0.32% by weight HPA-E. Optionally, the composition contains not more than about 0.06%, about 0.05%, or about 0.04%, by weight, DHA or derivative thereof such as ethyl-DHA. In one embodiment, the composition contains substantially no or no amount of DHA or derivative thereof such as ethyl-DHA. The composition further optionally comprises one or more antioxidants (e.g. tocopherol) in an amount of not more than about 0.5% or not more than 0.05%. In another embodiment, the composition comprises about 0.05% to about 0.4%, for example about 0.2% by weight tocopherol. In another embodiment, about 500 mg to about 1 g of the composition is provided in a capsule shell. In another embodiment, the capsule shell contains no chemically modified gelatin.

In another embodiment, compositions useful in accordance with the disclosure comprise, consist essentially of, or consist of at least 96% by weight ethyl eicosapentaenoate, about 0.22% to about 0.4% by weight ODTA-E, about 0.075% to about 0.20% by weight NDPA-E, about 0.25% to about 0.40% by weight AA-E, about 0.3% to about 0.4% by weight ETA-E and about 0.075% to about 0.25% by weight HPA-E. Optionally, the composition contains not more than about 0.06%, about 0.05%, or about 0.04%, by weight, DHA or derivative thereof such as ethyl-DHA. In one embodiment, the composition contains substantially no or no amount of DHA or derivative thereof such as ethyl-DHA. The composition further optionally comprises one or more antioxidants (e.g. tocopherol) in an amount of not more than about 0.5% or not more than 0.05%. In another embodiment, the composition comprises about 0.05% to about 0.4%, for example about 0.2% by weight tocopherol. In another embodiment, the disclosure provides a dosage form comprising about 500 mg to about 1 g of the foregoing composition in a capsule shell. In one embodiment, the dosage form is a gel- or liquid-containing capsule and is packaged in blister packages of about 1 to about 20 capsules per sheet.

In another embodiment, compositions useful in accordance with the disclosure comprise, consist essentially of, or consist of at least 96%, 97%, or 98%, by weight, ethyl eicosapentaenoate, about 0.25% to about 0.38% by weight ODTA-E, about 0.10% to about 0.15% by weight NDPA-E, about 0.25% to about 0.35% by weight AA-E, about 0.31% to about 0.38% by weight ETA-E, and about 0.08% to about 0.20% by weight HPA-E. Optionally, the composition contains not more than about 0.06%, about 0.05%, or about 0.04%, by weight, DHA or derivative thereof such as ethyl-DHA. In one embodiment, the composition contains substantially no or no amount of DHA or derivative thereof such as ethyl-DHA. The composition further optionally comprises one or more antioxidants (e.g. tocopherol) in an amount of not more than about 0.5% or not more than 0.05%. In another embodiment, the composition comprises about 0.05% to about 0.4%, for example about 0.2% by weight tocopherol. In another embodiment, the disclosure provides a dosage form comprising about 500 mg to about 1 g of the foregoing composition in a capsule shell. In another embodiment, the capsule shell contains no chemically modified gelatin.

In another embodiment, a composition as described herein is administered to a subject once or twice per day. In another embodiment, 1, 2, 3, or 4 capsules, each containing about 1 g of a composition as described herein, are administered to a subject daily. In another embodiment, 1 or 2 capsules, each containing about 1 g of a composition as described herein, are administered to the subject in the morning, for example between about 5 am and about 11 am, and 1 or 2 capsules, each containing about 1 g of a composition as described herein, are administered to the subject in the evening, for example between about 5 μm and about 11 pm.

In one embodiment, a subject being treated in accordance with methods of the disclosure is not on fibrate or nitrate therapy.

In another embodiment, compositions useful in accordance with methods of the disclosure are orally deliverable. The terms “orally deliverable” or “oral administration” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration. In one embodiment, the composition is present in a capsule, for example a soft gelatin capsule.

A composition for use in accordance with the disclosure can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e. 1 to about 10, 1 to 8, 1 to 6, 1 to 4, or 1 to 2) of times per day, or as many times as needed to elicit a therapeutic response.

In another embodiment, the disclosure provides use of any composition described herein for treating moderate to severe hypertriglyceridemia in a subject in need thereof, comprising: providing a subject having a fasting baseline triglyceride level of about 500 mg/dl to about 1500 mg/dl and administering to the subject a pharmaceutical composition as described herein. In one embodiment, the composition comprises about 1 g to about 4 g of eicosapentaenoic acid ethyl ester, wherein the composition contains substantially no docosahexaenoic acid.

### EXAMPLES

### Example 1. Safety and Efficacy of Ultra-Pure EPA

A multi-center, placebo-controlled, randomized, double-blind, 12-week study is performed to evaluate the efficacy and safety of >96% E-EPA in patients with fasting triglyceride levels ≥200 mg/dl and <500 mg/dl despite statin therapy (the mean of two qualifying entry values needs to be ≥185 mg/dl and at least one of the values needs to be ≥200 mg/dl). The primary objective of the study is to determine the efficacy of >96% E-EPA 2 g daily and 4 g daily, compared to placebo, in lowering fasting TG levels in patients with high risk for cardiovascular disease and with fasting TG levels ≥200 mg/dL and <500 mg/dL, despite treatment to LDL-C goal on statin therapy.

The secondary objectives of this study are the following:
1. To determine the safety and tolerability of >96% E-EPA 2 g daily and 4 g daily;
2. To determine the effect of >96% E-EPA on lipid and apolipoprotein profiles including TC, non-HDL-C, LDL-C, HDL-C, and VHDL-C;
3. To determine the effect of >96% E-EPA on Lp-PLA₂ from baseline to week 12;
4. To determine the effect of >96% E-EPA on LDL particle number and size;
5. To determine the effect of >96% E-EPA on oxidized LDL;
6. To determine the effect of >96% E-EPA on FPG and HbA_{1c};
7. To determine the effect of >96% E-EPA on insulin resistance;
8. To determine the effect of >96% E-EPA on hsCRP;
9. To determine the effects of >96% E-EPA 2 g daily and 4 g daily on the incorporation of fatty acids into red blood cell membranes and into plasma phospholipids;
10. To explore the relationship between baseline fasting TG levels and the reduction in fasting TG levels; and
11. To explore the relationship between changes of fatty acid concentrations in plasma and red blood cell membranes, and the reduction in fasting TG levels.

The population for this study is men and women >18 years of age with a body mass index ≤45 kg/m² with fasting TG levels greater than or equal to 200 mg/dl and less than 500 mg/dl and on a stable dose of statin therapy (with or without ezetimibe). The statin must be atorvastatin, rosuvastatin, or simvastatin. The dose of statin must be stable for ≥4 weeks prior to the LDL-C/TG baseline qualifying measurement for randomization. The statin dose will be optimal such that the patients are at their LDL-C goal at the LDL-C/TG baseline qualifying measurements. The same statin at the same dose will be continued until the study ends.

Patients taking any additional non-statin, lipid-altering medications (niacin >200 mg/day, fibrates, fish oil, other products containing omega-3 fatty acids, or other herbal products or dietary supplements with potential lipid-altering effects), either alone or in combination with statin therapy (with or without ezetimibe), must be able to safely discontinue non-statin, lipid-altering therapy at screening.

Patients at high risk for CVD, i.e., patients with clinical coronary heart disease (CHD) or clinical CHD risk equivalents (10-year risk >20%) as defined in the National Cholesterol Education Program (NCEP) Adult Treatment Panel III (ATP III) Guidelines will be eligible to participate in this study. Those include patients with any of the following criteria: (1) Known CVD, either clinical coronary heart disease (CHD), symptomatic carotid artery disease (CAD), peripheral artery disease (PAD), or abdominal aortic aneurism; or (2) Diabetes Mellitus (Type 1 or 2).

Approximately 648 patients will be randomized at approximately 80 centers in the U.S. The study will be a 18- to 20-week, Phase 3, multi-center study consisting of 2 study periods: (1) A 6- to 8-week screening period that includes a diet and lifestyle stabilization, a non-statin lipid-altering treatment washout, and an LDL-C and TG qualifying period and (2) A 12-week, double-blind, randomized, placebo-controlled treatment period.

During the screening period and double-blind treatment period, all visits are to be within ±3 days of the scheduled time. All patients will continue to take the statin product (with or without ezetimibe) at the same dose they were taking at screening throughout their participation in the study.

The 6- to 8-week screening period includes a diet and lifestyle stabilization, a non-statin lipid-altering treatment washout, and an LDL-C and TG qualifying period. The screening visit (Visit 1) will occur for all patients at either 6 weeks (for patients on stable statin therapy (with or without ezetimibe] at screening) or 8 weeks (for patients who will require washout of their current non-statin lipid-altering therapy at screening) before randomization, as follows:
Patients who do not require a washout: The screening visit will occur at Visit 1 (Week −6). Eligible patients will enter a 4-week diet and lifestyle stabilization period. At the screening visit, all patients will receive counseling regarding the importance of the National Cholesterol Education Program (NCEP) Therapeutic Lifestyle Changes (TLC) diet and will receive basic instructions on how to follow this diet.
Patients who will require a washout: The screening visit will occur at Visit 1 (Week −8). Eligible patients will begin a 6-week washout period at the screening visit (i.e. 6 weeks washout before the first LDL-C/TG qualifying visit). Patients will receive counseling regarding the NCEP TLC diet and will receive basic instructions on how to follow this diet. Site personnel will contact patients who do not qualify for participation based on screening laboratory test results to instruct them to resume their prior lipid-altering medications.

At the end of the 4-week diet and lifestyle stabilization period or the 6-week diet and stabilization and washout period, eligible patients will enter the 2-week LDL-C and TG qualifying period and will have their fasting LDL-C and TG levels measured at Visit 2 (Week −2) and Visit 3 (Week −1). Eligible patients must have an average fasting LDL-C level ≥40 mg/dL and <100 mg/dL and an average fasting TG level ≥200 mg/dL and <500 mg/dL to enter the 12-week double-blind treatment period. The LDL-C and TG levels for qualification will be based on the average (arithmetic mean) of the Visit 2 (Week −2) and Visit 3 (Week −1) values. If a patient's average LDL-C and/or TG levels from Visit 2 and Visit 3 fall outside the required range for entry into the study, an additional fasting lipid profile can be collected 1 week later at Visit 3.1. If a third sample is collected at Visit 3.1, entry into the study will be based on the average (arithmetic mean) of the values from Visit 3 and Visit 3.1.

After confirmation of qualifying fasting LDL-C and TG values, eligible patients will enter a 12-week, randomized, double-blind treatment period. At Visit 4 (Week 0), patients will be randomly assigned to 1 of the following treatment groups:
>96% E-EPA 2 g daily,
>96% E-EPA 4 g daily, or
Placebo.

Approximately 216 patients per treatment group will be randomized in this study. Stratification will be by type of statin (atorvastatin, rosuvastatin, or simvastatin), the presence of diabetes, and gender.

During the double-blind treatment period, patients will return to the site at Visit 5 (Week 4), Visit 6 (Week 11), and Visit 7 (Week 12) for efficacy and safety evaluations.

Eligible patients will be randomly assigned at Visit 4 (Week 0) to receive orally >96% E-EPA 2 g daily, >96% E-EPA 4 g daily, or placebo.

>96% E-EPA is provided in 1 g liquid-filled, oblong, gelatin capsules. The matching placebo capsule is filled with light liquid paraffin and contains 0 g of >96% E-EPA. >96% E-EPA capsules are to be taken with food (i.e. with or at the end of a meal).

During the double-blind treatment period, patients will take 2 capsules (>96% E-EPA or matching placebo) in the morning and 2 capsules in the evening for a total of 4 capsules per day.
Patients in the >96% E-EPA 2 g/day treatment group will receive 1 >96% E-EPA 1 g capsule and 1 matching placebo capsule in the morning and in the evening.
Patients in the >96% E-EPA 4 g/day treatment group will receive 2 >96% E-EPA 1 g capsules in the morning and evening.

Patients in the placebo group will receive 2 matching placebo capsules in the morning and evening.

The primary efficacy variable for the double-blind treatment period is percent change in TG from baseline to Week 12 endpoint. The secondary efficacy variables for the double-blind treatment period include the following:
Percent changes in total cholesterol (TC), high-density lipoprotein cholesterol (HDL-C), LDL-C, calculated non-HDL-C, and very low-density lipoprotein cholesterol (VLDL-C) from baseline to Week 12 endpoint;
Percent change in very low-density lipoprotein TG from baseline to Week 12;
Percent changes in apolipoprotein A-I (apo A-I), apolipoprotein B (apo B), and apo A-I/apo B ratio from baseline to Week 12;
Percent changes in lipoprotein(a) from baseline to Week 12;
Percent changes in LDL particle number and size, measured by nuclear magnetic resonance, from baseline to Week 12;
Percent change in remnant-like particle cholesterol from baseline to Week 12;
Percent change in oxidized LDL from baseline to Week 12;
Changes in FPG and HbA_{1c} from baseline to Week 12;
Change in insulin resistance, as assessed by the homeostasis model index insulin resistance, from baseline to Week 12;
Percent change in lipoprotein associated phospholipase A₂ (Lp-PLA₂) from baseline to Week 12;
Change in intracellular adhesion molecule-1 from baseline to Week 12;
Change in interleukin-2 from baseline to Week 12;
Change in plasminogen activator inhibitor-1 from baseline to Week 12. Note: this parameter will only be collected at sites with proper storage conditions;
Change in hsCRP from baseline to Week 12; and
Change in plasma concentration and red blood cell membrane content of fatty acid from baseline to Week 12 including EPA, DPA, DHA, AA, dihomo-γ-linolenic acid (DGLA), the ratio of EPA/AA, ratio of oleic acid/stearic acid (OA/SA), and the ratio of total omega-3 acids over total omega-6 acids.

Safety assessments will include adverse events, clinical laboratory measurements (chemistry, hematology, and urinalysis), 12-lead electrocardiograms (ECGs), vital signs, and physical examinations.

For TG, TC, HDL-C, LDL-C, calculated non-HDL-C, and VLDL-C, baseline will be defined as the average of Visit 4 (Week 0) and the preceding lipid qualifying visit (either Visit 3 [Week −1] or if it occurs, Visit 3.1) measurements. Baseline for all other efficacy parameters will be the Visit 4 (Week 0) measurement.

For TG, TC, HDL-C, LDL-C, calculated non-HDL-C, and VLDL-C, Week 12 endpoint will be defined as the average of Visit 6 (Week 11) and Visit 7 (Week 12) measurements.

Week 12 endpoint for all other efficacy parameters will be the Visit 7 (Week 12) measurement.

The primary efficacy analysis will be performed using a 2-way analysis of covariance (ANCOVA) model with treatment as a factor and baseline TG value as a covariate. The least-squares mean, standard error, and 2-tailed 95% confidence interval for each treatment group and for each comparison will be estimated. The same 2-way ANCOVA model will be used for the analysis of secondary efficacy variables.

The primary analysis will be repeated for the per-protocol population to confirm the robustness of the results for the intent-to-treat population.

Non-inferiority tests for percent change from baseline in LDL-C will be performed between >96% E-EPA doses and placebo using a non-inferiority margin of 6% and a significant level at 0.05.

For the following key secondary efficacy parameters, treatment groups will be compared using Dunnett's test to control the Type 1 error rate: TC, LDL-C, HDL-C, non-HDL-C, VLDL-C, Lp-PLA₂, and apo B. For the remaining secondary efficacy parameters, Dunnett's test will not be used and the ANCOVA output will be considered descriptive.

The evaluation of safety will be based primarily on the frequency of adverse events, clinical laboratory assessments, vital signs, and 12-lead ECGs. The primary efficacy variable is the percent change in fasting TG levels from baseline to Week 12. A sample size of 194 completed patients per treatment group will provide 90.6% power to detect a difference of 15% between >96% E-EPA and placebo in percent change from baseline in fasting TG levels, assuming a standard deviation of 45% in TG measurements and a significance level of p<0.05.

Previous data on fasting LDL-C show a difference in percent change from baseline of 2.2%, with a standard deviation of 15%, between study drug and placebo. A sample size of 194 completed patients per treatment group will provide 80% power to demonstrate non-inferiority (p<0.05, one-sided) of the LDL-C response between >96% E-EPA 4 g daily and placebo, within a 6% margin. To accommodate a 10% drop-out rate from randomization to completion of the double-blind treatment period, a total of 648 randomized patients is planned (216 patients per treatment group).

### Example 2. Improvement of Cognitive Performance in Subjects with Age-Associated Memory Impairment

A single-center, 6-week, double-blind, randomized, parallel-group, placebo-controlled, dose-ranging pilot study was performed to evaluate the efficacy, tolerability, and safety of >96% E-EPA in subjects with subjective and objective memory impairment according to generally accepted criteria for Age-Associated Memory Impairment (“AAMI”). The primary objective of the study was to determine the effect of >96% E-EPA 1 g, 2 g, and 4 g daily, compared to placebo, on cognitive performance in subjects with AAMI.

The secondary objectives of this study were the following:

1. To determine the effect of >96% E-EPA on the following tests in the computerized cognitive battery:
Continuity of attention tasks;
Quality of working memory tasks;
Quality of episodic memory tasks; and
Speed of attention tasks;

2. To determine the safety and tolerability of >96% E-EPA from routine clinical laboratory tests, adverse events (“AE”) monitoring, and vital signs; and

3. To determine the potential dose-effect relationship of >96% E-EPA on the cognitive endpoints by measurement of essential fatty acids in plasma and red blood cell membranes.

The population for this study was men and women between ages 50 and 70 with self-reported complaints of memory loss, subjective and objective cognitive impairment with a score of at least one standard deviation below that of the mean for age-matched elderly population as determined by the total score of between 13 and 20 from the Paired Associated Learning (“PAL”) subset of the Wechsler Memory Scale, evidence of adequate intellectual function as determined by a scaled score of at least 9 (raw score of at least 32) on the Vocabulary subtest of the Wechsler Adult Intelligence Scale and absence of dementia as determined by a score of 24 or higher on the Mini-Mental State Examination (“MMSE”).

Potential subjects were excluded based on the following exclusion criteria:
Unlikely or unable to comply with investigational medication dosing requirements;
Diagnosis of major depressive disorder, Alzheimer's or vascular dementia as defined according to the Mini International Neuropsychiatric Interview (“MINI”)/Diagnostic and Statistical Manual of Mental Disorders (4th edition) Text Revision (“TR”) criteria;
Past or current history of:
   a neurological or psychiatric disorder that could have affected cognitive function;
   inflammatory gastrointestinal disease such as Crohn's Disease or ulcerative colitis;
   cancer other than basal cell carcinoma;
   clinically significant cardiac abnormality as measured by 12-lead ECG;
Any other medical condition or intercurrent illness not adequately controlled, which, in the opinion of the study investigator, may have put the subject at risk when participating in the study or may have influenced the results of the study or affected the subject's ability to take part in the study;
Clinically significant abnormal screening results (e.g., haematology, biochemistry) on screening or vital signs that fell outside the normal range for this population, which in the opinion of the study investigator affected the subject's suitability for the study;
Changes to prescribed medication for a medical condition within 4 weeks of the baseline visit;
Omega-3 supplementation within 4 weeks of the baseline visit or during the study treatment period;
Currently taking anticoagulants or daily dose of aspirin greater than 325 mg.
Cough or flu remedies containing opiates or antihistamines within 2 weeks of the baseline visit or during the 6-week treatment period; and
Known allergy to any ingredients of the study drug or placebo.

Ninety-four subjects were randomized into one of six groups: 1 g E-EPA daily (n=23), 2 g E-EPA daily (n=24), 4 g E-EPA daily (n=24), 1 g placebo daily (n=7), 2 g placebo daily (n=8), and 4 g placebo daily (n=8). E-EPA was provided as 500 mg soft gel capsules containing >96% E-EPA and 0.2% dl-α-tocopherol as an antioxidant. Placebo capsules contained 467 mg of liquid paraffin and 0.2% dl-α-tocopherol. Ninety-one subjects completed the study. Two subjects in the 2 g E-EPA group and one subject in the 2 g placebo group discontinued the study.

The study consisted of a screening visit, a training visit, and four study visits. At the screening visit, subjects' eligibility was determined through cognitive tests (verbal paired associated learning [PAL] subscale, vocabulary subtest, Memory Assessment Clinics Questionnaire [MAC-Q], mini mental state evaluation [MMSE] and MINI [mini international neuropsychiatric interview; sections 1 and 2 of Diagnostic and Statistical Manual of Mental Disorders, 4th Edition (DSM-IV) plus dysthymia), haematology, clinical chemistry, and 12-lead electrocardiogram (ECG). At the training visit, subjects were shown how to use the CDR computerized system. Subjects took study drug for 6 weeks and on Days 0, 14, 28 and 42, subjects underwent the CDR cognitive test battery.

At screening cognitive testing and suitability for the study were assessed using the Verbal Paired Associates 1 (Wechsler Memory Scale), Vocabulary Subtest of the WAIS, MAC-Q, MMSE, and MINI (DSM-IV Sections 1 and 2 plus Dysthymia).

A selection of tasks from the CDR computerized cognitive assessment system were administered at Visit 2 (training visit), Visit 3 (baseline), Visit 4 (Day 14), Visit 5 (Day 28) and Visit 6 (Day 42). Parallel forms of the tests were presented at each testing session. All tasks were computer-controlled, the information presented on high resolution monitors, and the responses recorded via a response model containing two buttons: one marked ‘no’ and the other ‘yes’. Five CDR composite scores were used as the primary/secondary outcome variables. The task titles were:
Word Presentation
Immediate Word Recall
Picture Presentation
Simple Reaction Time
Digit Vigilance
Choice Reaction Time
Spatial Working Memory
Numeric Working Memory
Delayed Word Recall
Word Recognition
Picture Recognition
Bond-Lader Visual Analogue Scales of Mood and Alertness
Screen, Using the Computer Mouse

To ensure consistency of approach, full training on the cognitive tests and CDR test battery was provided to study site staff and study subjects. The results of each variable were automatically recorded using the machine interface developed by CDR.

Blood samples (10 mL) were collected at Visit 1 (screening) and at Visits 4, 5, and 6. Analysis was performed by MSR Lipid Analysis, Scottish Crop Research Institute, Dundee, UK. The screening sample acted as baseline for the EFA measurements. Lipid was extracted from plasma, serum, and RBC suspensions and converted into fatty acid methyl esters which were analyzed by gas chromatography to give fatty acid profiles as micrograms fatty acid per gram of sample (mFA/g) and normalized area percent.

All randomized subjects with at least 1 visit post-baseline were included in the Intent to Treat (“ITT”) population, regardless of treatment actually received.

All randomized subjects that completed the study, excluding significant protocol deviators, were defined as the Safety Per Protocol population. An Efficacy Per Protocol population was based on the Efficacy completers. The intercept of the Safety and Efficacy Per Protocol populations defined the Study Per Protocol Population.

All randomized subjects that received at least 1 dose of study medication were included in the Safety Population.

Summary statistics were provided for the ITT and Study Per Protocol Populations separately for all composite scores, major and supportive variables. Summary statistics were performed for both the unadjusted and difference from baseline data (i.e. the difference from the time matched predose assessments on Day 0). Summary statistics were calculated by treatment, day, and time-point. The summary statistics comprised n, mean, median, SD, standard error of mean (“SEM”), minimum, and maximum values.

Difference from baseline data for each major variable was evaluated by an Analysis of Covariance (“ANCOVA”) using SAS® PROC MIXED Version 8.2. Fixed effects for treatment, day, time point, treatment by day, treatment by time point, and treatment by day by time-point were fitted. Subject within treatment was fitted as a repeated effect using the repeated statement. The compound symmetry covariance structure was used. Subjects' time-matched predose assessments on Day 0 were used as a covariate in the analysis.

Least squares means (LS means) were calculated for treatment by day, treatment by time-point, and treatment by day by time-point interaction. This formal analysis was conducted for the ITT and Study PP Populations separately.

Safety evaluations were based on the safety population. Safety and tolerability were assessed in terms of AEs, vital signs, 12-lead ECG, clinical laboratory data, medical history, and study drug compliance. Safety and tolerability data were presented by treatment group.

RBC and plasma EFA data were collected at baseline, Day 14, 28, and 42 and summarized by visit for each treatment group. Change from baseline and percent change from baseline were also summarized. ANCOVA comparison of E-EPA dose groups and E-EPA versus placebo was performed.

### Efficacy Results.

All CDR cognitive test battery analyses were completed for the ITT and Study PP analysis populations.

For the Intent-to-Treat Analysis for Power of Attention, there was no statistically significant effect of treatment, nor any treatment by day, treatment by time-point, or treatment by day by time-point interactions. There was no LS mean difference between active treatment and placebo at any time-point.

For the contributing subtasks Simple Reaction Time and Digit Vigilance Speed, there were no statistically significant effects of treatment, nor any treatment by day, treatment by time-point, or treatment by day by time-point interactions. For the subtask measure Choice Reaction Time, there was a statistically significant treatment by day interaction (p=0.011).

For the Study Per-Protocol Power of Attention, there was no statistically significant effect of treatment, nor any treatment by day, treatment by time-point, or treatment by day by time-point interactions. There was no difference between active treatment and placebo at any time-point.

For the subtasks Simple Reaction Time and Digit Vigilance Speed, there were no statistically significant effects of treatment, nor any treatment by day, treatment by time-point, or treatment by day by time-point interactions. For the subtask measure, Choice Reaction Time, there was a statistically significant treatment by day interaction (p=0.013).

The Intent-to-Treat Continuity of Attention and the contributing subtask Digit Vigilance Targets Detected tests showed no statistically significant effect of treatment, nor any treatment by day, treatment by time-point, or treatment by day by time-point interactions.

For the Study Per Protocol Continuity of Attention test, there was no statistically significant effect of treatment, nor any treatment by day, treatment by time-point, or treatment by day by time-point interactions.

For the subtask Digit Vigilance Targets Detected, there was a statistically significant treatment by time-point interaction (p=0.040).

For the Intent-to-Treat Quality of Working Memory test, there was a statistically significant treatment by day interaction (p=0.019).

For the contributing subtask Spatial Working Memory Sensitivity Index, there was a statistically significant treatment by day interaction (p=0.015).

For Numeric Working Memory Sensitivity Index, there was a statistical trend for a treatment by day interaction (p=0.089).

For the Study Per-Protocol Quality of Working Memory test, there was a statistically significant treatment by day interaction (p=0.021).

For the contributing subtask Spatial Working Memory Sensitivity Index, there was a statistically significant treatment by day interaction (p=0.014).

For the Intent-to-Treat Quality of Episodic Secondary Memory test, there was no statistically significant effect of treatment, nor any treatment by day, treatment by time-point, or treatment by day by time-point interactions. The LS mean differences showed overall statistically significant decreases versus placebo for E-EPA 1 g and 2 g (p=0.040 and p=0.035, respectively).

For the contributing subtasks Immediate and Delayed Word Recall Accuracies and for Word and Picture Recognition Sensitivity Indices, there were no statistically significant effects of treatment or treatment by day, treatment by time-point, or treatment by day by time-point interactions. For Immediate Word Recall Accuracy, the LS mean differences showed statistically significant decreases for 1 g on Day 14 (p=0.028) and for 2 g on Day 28 (p=0.017). There were statistically significant decreases versus placebo for 1 g and 2 g at AM 1 (p=0.040 and p=0.028, respectively). There were statistically significant decreases for E-EPA 1 g versus placebo on Day 14 at PM 2 (p=0.020) and for 2 g on Day 28 at AM 1 (p=0.006). For Word Recognition Sensitivity Index, the LS mean differences showed statistically significant decreases for E-EPA 1 g on Day 28 (p=0.024) and for 4 g on Day 42 (p=0.038) versus placebo. There was a statistically significant decrease for 4 g at PM 2 (p=0.045) and a statistically significant decrease for 4 g versus placebo on Day 28 at PM 2 (p=0.030). For Picture Recognition Sensitivity Index, the LS mean differences showed statistically significant decrease for 1 g versus placebo on Day 28 at AM 2 (p=0.017) and at PM 2 (p=0.040). For the Study Per-Protocol Quality of Episodic Secondary Memory test, there were no statistically significant effects of treatment, nor treatment by day, treatment by time-point, or treatment by day by time-point interactions. The LS mean differences showed overall statistically significant decreases versus placebo for 1 g and 2 g (p=0.043 and p=0.036, respectively).

For the contributing subtasks Immediate and Delayed Word Recall Accuracies and for Word and Picture Recognition Sensitivity Indices, there were no statistically significant effects of treatment or treatment by day, treatment by time-point, or treatment by day by time-point interactions. For Immediate Word Recall Accuracy, the LS mean differences to placebo showed statistically significant decreases for E-EPA 1 g on Day 14 (p=0.024) and for 2 g on Day 28 (p=0.017). There were statistically significant decreases for 1 g and 2 g at AM 1 (p=0.038 and p=0.029, respectively) and for 1 g at AM 2 (p=0.048). There were statistically significant decreases for 1 g versus placebo on Day 14 at PM 2 (p=0.019) and for 2 g on Day 28 at AM 1 (p=0.006).

For Word Recognition Sensitivity Index, the LS mean differences to placebo showed statistically significant decreases for 4 g on Day 42 (p=0.038) and for 1 g on Day 28 (p=0.027).

For Picture Recognition Sensitivity Index, the LS mean differences showed statistically significant decreases versus placebo for 1 g on Day 28 at AM 2 (p=0.020) and PM 2 (p=0.026).

For Intent-to-Treat Speed of Memory and the contributing subtasks Spatial and Numeric Working Memory Speeds and Word, and Picture Recognition Speeds, there were no statistically significant effects of treatment, nor treatment by day, treatment by time-point, or treatment by day by time-point interactions. For Spatial Working Memory Speed, the LS mean differences showed a statistically significant benefit versus placebo for E-EPA 4 g on Day 14 at PM 1 (p=0.048) and a trend for a benefit for 4 g on Day 42 at AM 1 (p=0.061). For Picture Recognition Speed, there were trends for benefits versus placebo for 1 g on Day 14 at AM 2 (p=0.084) and on Day 28 at AM 1 (p=0.085).

For Study Per-Protocol Speed of Memory and the contributing subtasks Spatial and Numeric Working Memory Speed and Word, and Picture Recognition Speed, there were no statistical significant effects of treatment, nor any treatment by day, treatment by time-point, or treatment by day by time-point interactions.

For Intent-to-Treat Self-Rated Alertness, there was no statistically significant effect of treatment, nor any treatment by day, treatment by time-point, or treatment by day by time-point interactions. The LS mean differences showed a statistically significant decrease in ratings for E-EPA 2 g on Day 28 (p=0.047) versus placebo. There was a statistically significant decrease in ratings versus placebo for 2 g on Day 28 at AM 2 (p=0.041). For Study Per-Protocol Self-Rated Alertness, there was no statistically significant effect of treatment, nor any treatment by day, treatment by time-point, or treatment by day by time-point interactions. The LS mean differences showed a statistically significant decrease in ratings for E-EPA 2 g on Day 28 (p=0.035) versus placebo. There was a statistically significant decrease in ratings versus placebo for 2 g on Day 28 at AM 2 (p=0.033).

For Intent-to-Treat Self-Rated Contentment, there was a statistically significant treatment by day interaction (p<0.001). The LS mean difference to placebo showed no statistically significant effects. For Study Per-Protocol Self-Rated Contentment, there was a statistically significant treatment by day interaction (p<0.001). The LS mean difference to placebo showed no statistically significant effects.

For Intent-to-Treat Self-Rated Calmness, there was no statistically significant effect of treatment, nor any treatment by day, treatment by time-point or treatment by day by time-point interactions. For Study Per-Protocol Self-Rated Calmness, there was no statistically significant effect of treatment, nor any treatment by day, treatment by time-point, or treatment by day by time-point interactions. The LS mean differences showed a statistical trend for an increase in ratings versus placebo for E-EPA 4 g on Day 42 at PM 1 (p=0.071).

A post-hoc analysis compared the individual placebo groups (1 g, 2 g, and 4 g paraffin oil) with the corresponding E-EPA doses.

The pattern of data provided evidence that E-EPA 4 g might improve speed in the attention based measures. For Power of Attention, there was an overall benefit versus the corresponding placebo for 4 g on Day 42. The subtask Simple Reaction Time showed improvements in performance for 4 g at PM 2 collapsed across days and at several time-points on Days 14 and 42. The improvements for 4 g were most pronounced in the Choice Reaction Time task, where there was an overall benefit versus corresponding placebo for 4 g, reflecting a benefit for 4 g over placebo on all study days. The pattern of improvement in performance throughout the assessment days was quite convincing as the improvements began on Day 14 with improvements seen at 2 time points, whereas on Day 42 E-EPA 4 g was superior to placebo at every time point.

For Continuity of Attention, there were isolated declines or improvements in performance, but there was no general pattern of effects and it was considered unlikely these differences were due to the study compound. For Quality of Working Memory and in the subtask measure Numeric Working Memory Sensitivity Index, there were, as in the original analyses, only isolated improvements and declines in performance that were most likely not treatment-related. However, for Spatial Working Memory Sensitivity Index, there was an overall benefit for E-EPA 4 g over placebo on Day 42 in the Study PP Population, which corresponds to the improvements seen for the attention based measures.

For Quality of Episodic Secondary Memory and contributing subtasks, there were a number of decreases for E-EPA that could be explained by the pre-existing differences in performance between the placebo and active treatment groups which was seen in the original analyses. In contrast to the original analysis, the subtask measures of Speed of Memory showed some signs of improvement in performance for active treatment, mostly for 1 g versus placebo. For Self-rated Alertness and Self-rated Contentment, the 1 g dose showed decreases in ratings on Days 14 and 28. However, these decreases were not correlated with a decline in performance in the CDR attention tasks. As with the original planned analysis, there were no differences between active treatment and placebo in Self-Rated Calmness.

### Safety Results.

Subjects who used less than 80% of the prescribed dose were to be considered non-compliant; other than those subjects who withdrew for other reasons only, 1 subject fell into this category and was withdrawn.

Overall, 139 treatment emergent AEs (“TEAEs”) were reported by 62 (66.0%) of subjects during the study. Most TEAEs were considered mild in severity and unrelated to study drug. More TEAEs were reported for the E-EPA treatment groups (105 events) compared to the placebo treatment groups (34 events). One SAE was reported for the E-EPA 2 g treatment group and 3 subjects discontinued due to TEAEs: 2 subjects from the E-EPA 2 g treatment group (the primary reason for discontinuation for 1 of these subjects was non-compliance), and 1 subject from the placebo 2 g treatment group.

There were no deaths during the study. No TEAEs were Definitely Related to the study drug. One subject receiving 1 g E-EPA experienced nausea that was Probably Related to the study drug. Another subject receiving 4 g E-EPA experienced diarrhea that was Probably Related to the study drug; another subject receiving 2 g placebo also experienced diarrhea that was Probably Related to the study drug. Five subjects experienced nausea that was Possibly Related to the study drug; two were in the 1 g E-EPA cohort; one was in the 2 g E-EPA cohort; two were in the 4 g E-EPA cohort. One subject receiving 2 g placebo experienced headache that was Possibly Related to the study drug. All other TEAEs were Not Related or Unlikely Related to the study drug, and included nasopharyngitis (n=3), cystitis (n=2), cough (n=7), toothache (n=2), pharyngolaryngeal pain (n=2), back pain (n=2), pollakiuria (n=2), influenza-like illness (n=2), headache (n=15), diarrhea (n=2), and nausea (n=1).

One subject with a history of transient ischaemic attack, hypertension, and osteoarthritis of the hand and osteopaenia receiving 2 g E-EPA experienced worsening epigastric chest pain 17 days after the start of the study and 9 days after the last dose of the study drug. A planned endoscopy revealed oesophagitis and a small hiatus hernia. The subject was treated with omeprazole, which settled her symptoms. The subject had taken felodipine, rosuvastatin, aspirin, glucosamine, and quinine within 14 days of the onset of her symptoms. The study investigator determined that her symptoms were unrelated to the study drug and withdrew the subject from the study. No other Serious Adverse Events occurred during the study.

Essential fatty acid parameters in plasma and RBCs were measured at baseline and on Day 14, 28, and 48 (shown in Tables 1-6). Notable changes for these parameters occurred in the E-EPA treatment groups at Days 14, 28, and 42 compared to placebo. EPA, DPAn-3, and EPA/AA ratio values increased substantially from baseline, in plasma and RBC, to Day 42 for the E-EPA 1, 2, and 4 g treatment groups, but remained similar to baseline in the placebo treatment groups. AA, DHA, and DGLA values decreased substantially from baseline, in plasma and RBC, to day 42 for the E-EPA 1, 2, and 4 g treatment groups, but remained similar to baseline in the placebo treatment groups. The difference in EPA, AA (RBC only), DPAn-3, DGLA (1 g only for plasma), and EPA/AA ratio levels in the plasma and RBC were significantly (LS means, p<0.05) different for the E-EPA 4 g treatment group compared to the E-EPA 1 g and 2 g treatment groups.

TABLE 1
EFA Parameter EPA (Plasma and RBC) Mean change from Baseline to Days 14, 28 and 42.

| EFA | Ethyl-EPA | | | Placebo | | |
|---|---|---|---|---|---|---|
| Parameter | 1 g | 2 g | 4 g | 1 g | 2 g | 4 g |
| (μg/g) | (N = 23) | (N = 24) | (N = 24) | (N = 7) | (N = 8) | (N = 8) |

Plasma

| Baseline: n | 23 | 24 | 24 | 7 | 8 | | 8 | |
|---|---|---|---|---|---|---|---|---|
| Mean (SD) | 48.3 (31.03) | 44.9 (25.01) | 49.1 (17.23) | 47.5 (26.41) | 42.1 | (16.18) | 42.5 | (11.86) |
| Day 14: n | 23 | 22 | 24 | 7 | 7 | | 8 | |
| Mean (SD) | 61.2 (26.61) | 124.6 (42.25) | 207.7 (57.05) | 1.6 (24.69) | −1.2 | (19.82) | 21.9 | (32.91) |
| Day 28: n | 22 | 22 | 24 | 7 | 7 | | 8 | |
| Mean (SD) | 60.3 (36.03) | 142.2 (46.23) | 215.2 (58.68) | 6.5 (15.46) | 1.6 | (13.64) | 1.3 | (14.03) |
| Day 42: n | 23 | 22 | 24 | 7 | 7 | | 8 | |
| Mean (SD) | 62.0 (39.43) | 133.4 (43.34) | 204.6 (80.69) | 11.9 (26.34) | 0.4 | (21.18) | 4.4 | (23.32) |

1 or 2 g versus 4 g

| | | | | | | |
|---|---|---|---|---|---|---|
| LS Mean | −111.8 | −60.9 | — | — | — | — |
| CI | −123.6, −100 | −7.27, −49.0 | — | — | — | — |
| p-value | <0.001 | <0.001 | | | | |

RBC

| Baseline: n | 23 | 24 | 24 | 7 | 7 | | 8 | |
|---|---|---|---|---|---|---|---|---|
| Mean (SD) | 19.8 (10.85) | 18.9 (8.91) | 19.8 (5.28) | 20.4 (5.77) | 19.3 | (6.58) | 17.2 | (4.94) |
| Day 14: n | 23 | 22 | 24 | 7 | 7 | | 8 | |
| Mean (SD) | 12.3 (7.39) | 26.9 (9.15) | 39.5 (13.16) | −0.5 (6.32) | 0.0 | (7.17) | 2.6 | (6.73) |
| Day 28: n | 22 | 22 | 24 | 7 | 7 | | 8 | |
| Mean (SD) | 14.5 (10.47) | 32.9 (10.11) | 50.2 (15.82) | 1.5 (4.16) | 0.0 | (7.06) | 0.6 | (4.42) |
| Day 42: n | 23 | 22 | 24 | 7 | 7 | | 8 | |
| Mean (SD) | 17.6 (11.89) | 38.3 (12.46) | 52.5 (20.56) | −0.2 (5.90) | 1.0 | (8.01) | −0.2 | (6.97) |

1 or 2 g versus 4 g

| | | | | | | |
|---|---|---|---|---|---|---|
| LS Mean | −24.4 | −11.8 | — | — | — | — |
| CI | −27.6, −21.2 | −15.0, −8.6 | — | — | — | — |
| p-value | <0.001 | <0.001 | | | | |

TABLE 2
EFA Parameter AA (Plasma and RBC) Mean change from Baseline to Days 14, 28 and 42.

| EFA | Ethyl-EPA | | | Placebo | | |
|---|---|---|---|---|---|---|
| Parameter | 1 g | 2 g | 4 g | 1 g | 2 g | 4 g |
| (μg/g) | (N = 23) | (N = 24) | (N = 24) | (N = 7) | (N = 8) | (N = 8) |

Plasma

| Baseline: n | 23 | | 24 | | 24 | 7 | | 8 | | 8 | |
|---|---|---|---|---|---|---|---|---|---|---|---|
| Mean (SD) | 202.5 | (44.40) | 227.3 | (42.26) | 220.9 (42.80) | 210.7 | (35.68) | 191.6 | (28.24) | 248.0 | (53.52) |
| Day 14: n | 23 | | 22 | | 24 | 7 | | 7 | | 8 | |
| Mean (SD) | −9.7 | (22.20) | −13.9 | (22.13) | −27.2 (28.89) | 0.8 | (40.00) | −14.4 | (19.45) | −5.9 | (25.00) |
| Day 28: n | 22 | | 22 | | 24 | 7 | | 7 | | 8 | |
| Mean (SD) | −11.3 | (28.13) | 21.6 | (28.32) | −43.7 (32.24) | 3.8 | (28.11) | −7.4 | (23.72) | −16.4 | (31.42) |
| Day 42: n | 23 | | 22 | | 24 | 7 | | 7 | | 8 | |
| Mean (SD) | −8.7 | (31.35) | −27.3 | (26.76) | −48.3 (22.20) | 8.2 | (20.30) | −11.5 | (20.88) | −11.0 | (25.82) |

1 or 2 g versus 4 g

| | | | | | | |
|---|---|---|---|---|---|---|
| LS Mean | 4.2 | 15.6 | — | — | — | — |
| CI | −8.0, 16.4 | 3.4, 27.8 | — | — | — | — |
| p-value | 0.496 | 0.013 | | | | |

RBC

| Baseline: n | 23 | | 24 | | 24 | 7 | | 8 | | 8 | |
|---|---|---|---|---|---|---|---|---|---|---|---|
| Mean (SD) | 171.2 | (19.79) | 172.8 | (22.79) | 171.0 (25.17) | 176.4 | (17.65) | 152.8 | (17.36) | 180.4 | (23.68) |
| Day 14: n | 23 | | 22 | | 24 | 7 | | 7 | | 8 | |
| Mean (SD) | −8.1 | (21.95) | −3.1 | (25.84) | −15.7 (26.76) | −8.5 | (22.75) | 3.0 | (18.20) | −8.1 | (27.53) |
| Day 28: n | 22 | | 22 | | 24 | 7 | | 7 | | 8 | |
| Mean (SD) | −17.0 | (20.69) | −14.1 | (26.89) | −22.8 (29.56) | 5.2 | (22.95) | −2.6 | (17.78) | −8.2 | (26.89) |
| Day 42: n | 23 | | 22 | | 24 | 7 | | 7 | | 8 | |
| Mean (SD) | −14.2 | (27.69) | −18.8 | (25.62) | −34.4 (31.44) | −9.8 | (21.59) | 9.7 | (16.58) | −10.6 | (33.49) |

1 or 2 g versus 4 g

| | | | | | | |
|---|---|---|---|---|---|---|
| LS Mean | 8.4 | 9.8 | — | — | — | — |
| CI | 2.0, 14.9 | 3.3, 16.2 | — | — | — | — |
| p-value | 0.010 | 0.003 | | | | |

TABLE 3
EFA Parameter DHA (Plasma and RBC) Mean change from Baseline to Days 14, 28 and 42.

| EFA | Ethyl-EPA | | | Placebo | | |
|---|---|---|---|---|---|---|
| Parameter | 1 g | 2 g | 4 g | 1 g | 2 g | 4 g |
| (μg/g) | (N = 23) | (N = 24) | (N = 24) | (N = 7) | (N = 8) | (N = 8) |

Plasma

| Baseline: n | 23 | | 24 | | 24 | | 7 | | 8 | | 8 | |
|---|---|---|---|---|---|---|---|---|---|---|---|---|
| Mean (SD) | 73.1 | (30.43) | 75.1 | (24.02) | 78.8 | (19.00) | 73.7 | (14.21) | 73.3 | (27.74) | 76.7 | (15.68) |
| Day 14: n | 23 | | 22 | | 24 | | 7 | | 7 | | 8 | |
| Mean (SD) | −6.4 | (13.30) | −5.4 | (14.29) | −10.3 | (13.35) | 0.4 | (18.86) | −0.8 | (14.28) | 13.8 | (21.05) |
| Day 28: n | 22 | | 22 | | 24 | | 7 | | 7 | | 8 | |
| Mean (SD) | −6.6 | (15.53) | −8.1 | (15.82) | −13.5 | (14.10) | 4.7 | (16.31) | −0.6 | (8.29) | 6.0 | (17.36) |
| Day 42: n | 23 | | 22 | | 24 | | 7 | | 7 | | 8 | |
| Mean (SD) | −5.4 | (18.17) | −6.0 | (16.69) | −13.8 | (15.31) | 11.8 | (21.27) | 0.8 | (17.57) | 6.2 | (13.40) |

1 or 2 g versus 4 g

| | | | | | | |
|---|---|---|---|---|---|---|
| LS Mean | −0.8 | 1.5 | — | — | — | — |
| CI | −7.3, 5.7 | −5.0, 8.1 | — | — | — | — |
| p-value | 0.810 | 0.644 | | | | |

RBC

| Baseline: n | 23 | | 24 | | 24 | | 7 | | 8 | | 8 | |
|---|---|---|---|---|---|---|---|---|---|---|---|---|
| Mean (SD) | 66.5 | (18.65) | 64.8 | (17.65) | 68.3 | (14.24) | 71.1 | (7.48) | 66.0 | (15.90) | 66.2 | (15.83) |
| Day 14: n | 23 | | 22 | | 24 | | 7 | | 7 | | 8 | |
| Mean (SD) | −4.6 | (9.76) | −2.0 | (9.46) | −6.9 | (9.13) | −5.5 | (11.93) | −0.2 | (12.39) | −0.4 | (12.50) |
| Day 28: n | 22 | | 22 | | 24 | | 7 | | 7 | | 8 | |
| Mean (SD) | −6.4 | (11.57) | −6.2 | (9.34) | −8.7 | (11.63) | 0.6 | (12.86) | −0.3 | (11.29) | 1.1 | (12.54) |
| Day 42: n | 23 | | 22 | | 24 | | 7 | | 7 | | 8 | |
| Mean (SD) | −7.0 | (12.20) | −6.3 | (9.42) | −13.8 | (13.76) | −4.1 | (12.02) | 4.6 | (12.94) | −0.1 | (17.63) |

1 or 2 g versus 4 g

| | | | | | | |
|---|---|---|---|---|---|---|
| LS Mean | 1.0 | 1.0 | — | — | — | — |
| CI | −3.5, 5.4 | −3.5, 5.5 | — | — | — | — |
| p-value | 0.674 | 0.664 | | | | |

TABLE 4
EFA Parameter DPAn-3 (Plasma and RBC) Mean change from Baseline to Days 14, 28 and 42.

| EFA | Ethyl-EPA | | | Placebo | | |
|---|---|---|---|---|---|---|
| Parameter | 1 g | 2 g | 4 g | 1 g | 2 g | 4 g |
| (μg/g) | (N = 23) | (N = 24) | (N = 24) | (N = 7) | (N = 8) | (N = 8) |

Plasma

| Baseline: n | 23 | | 24 | | 24 | | 7 | | 8 | | 8 | |
|---|---|---|---|---|---|---|---|---|---|---|---|---|
| Mean (SD) | 21.1 | (6.62) | 19.7 | (4.50) | 21.7 | (4.69) | 17.9 | (5.18) | 18.0 | (4.39) | 19.0 | (2.67) |
| Day 14: n | 23 | | 22 | | 24 | | 7 | | 7 | | 8 | |
| Mean (SD) | 7.5 | (5.11) | 17.4 | (7.49) | 24.5 | (11.28) | −0.2 | (3.13) | −1.0 | (3.59) | 2.2 | (4.98) |
| Day 28: n | 22 | | 22 | | 24 | | 7 | | 7 | | 8 | |
| Mean (SD) | 8.9 | (5.62) | 19.4 | (8.48) | 29.7 | (13.23) | 1.2 | (2.06) | 0.6 | (3.44) | 1.3 | (3.40) |
| Day 42: n | 23 | | 22 | | 24 | | 7 | | 7 | | 8 | |
| Mean (SD) | 11.3 | (6.61) | 19.3 | (8.63) | 32.0 | (16.01) | 2.2 | (3.29) | 0.1 | (3.61) | 0.8 | (6.70) |

1 or 2 g versus 4 g

| | | | | | | |
|---|---|---|---|---|---|---|
| LS Mean | −15.1 | −9.5 | — | — | — | — |
| CI | −17.6, −12.7 | −12.0, −7.1 | — | — | — | — |
| p-value | <0.001 | <0.001 | | | | |

RBC

| Baseline: n | 23 | | 24 | | 24 | | 7 | | 8 | | 8 | |
|---|---|---|---|---|---|---|---|---|---|---|---|---|
| Mean (SD) | 34.1 | (5.43) | 33.2 | (4.51) | 34.5 | (4.34) | 34.0 | (4.27) | 33.0 | (1.20) | 32.4 | (2.41) |
| Day 14: n | 23 | | 22 | | 24 | | 7 | | 7 | | 8 | |
| Mean (SD) | 0.9 | (5.03) | 5.6 | (6.28) | 5.4 | (5.38) | −2.8 | (4.86) | −0.3 | (4.96) | −0.9 | (4.74) |
| Day 28: n | 22 | | 22 | | 24 | | 7 | | 7 | | 8 | |
| Mean (SD) | 3.3 | (5.42) | 9.4 | (6.74) | 12.4 | (6.98) | 0.1 | (4.51) | −0.8 | (4.03) | −0.6 | (5.19) |
| Day 42: n | 23 | | 22 | | 24 | | 7 | | 7 | | 8 | |
| Mean (SD) | 6.5 | (6.19) | 13.2 | (7.23) | 16.2 | (10.07) | −1.8 | (4.64) | 2.2 | (4.44) | −0.9 | (6.03) |

1 or 2 g versus 4 g

| | | | | | | |
|---|---|---|---|---|---|---|
| LS Mean | −6.2 | −2.5 | — | — | — | — |
| CI | −7.8, −4.7 | −4.1, −1.0 | — | — | — | — |
| p-value | <0.001 | 0.002 | | | | |

TABLE 5
EFA Parameter DGLA (Plasma and RBC) Mean change from Baseline to Days 14, 28 and 42.

| EFA | Ethyl-EPA | | | Placebo | | |
|---|---|---|---|---|---|---|
| Parameter | 1 g | 2 g | 4 g | 1 g | 2 g | 4 g |
| (μg/g) | (N = 23) | (N = 24) | (N = 24) | (N = 7) | (N = 8) | (N = 8) |

Plasma

| Baseline: n | 23 | | 24 | | 24 | | 7 | | 8 | | 7 | |
|---|---|---|---|---|---|---|---|---|---|---|---|---|
| Mean (SD) | 51.2 | (15.01) | 53.5 | (14.12) | 57.1 | (14.73) | 51.6 | (9.20) | 41.6 | (10.30) | 52.6 | (7.74) |
| Day 14: n | 23 | | 22 | | 24 | | 7 | | 7 | | 8 | |
| Mean (SD) | −10.4 | (10.90) | −14.1 | (6.88) | −22.9 | (9.00) | −4.1 | (8.07) | −0.0 | (8.63) | −1.0 | (11.58) |
| Day 28: n | 22 | | 22 | | 24 | | 7 | | 7 | | 8 | |
| Mean (SD) | −10.6 | (10.23) | −16.2 | (9.88) | −24.2 | (10.73) | −4.6 | (7.43) | −0.6 | (5.91) | 1.5 | (11.78) |
| Day 42: n | 23 | | 22 | | 24 | | 7 | | 7 | | 8 | |
| Mean (SD) | −9.4 | (9.41) | −17.3 | (9.92) | −22.5 | (10.87) | −3.9 | (12.90) | 0.9 | (9.34) | 0.8 | (11.04) |

1 or 2 g versus 4 g

| | | | | | | |
|---|---|---|---|---|---|---|
| LS Mean | 3.7 | 2.5 | — | — | — | — |
| CI | 0.4, 7.0 | −0.9, 5.8 | — | — | — | — |
| p-value | 0.028 | 0.143 | | | | |

RBC

| Baseline: n | 23 | | 24 | | 24 | | 7 | | 8 | | 7 | |
|---|---|---|---|---|---|---|---|---|---|---|---|---|
| Mean (SD) | 23.0 | (5.19) | 23.0 | (5.76) | 24.0 | (5.77) | 22.4 | (5.06) | 19.7 | (5.87) | 22.4 | (4.91) |
| Day 14: n | 23 | | 22 | | 24 | | 7 | | 7 | | 8 | |
| Mean (SD) | −2.7 | (3.82) | −2.6 | (3.54) | −5.3 | (4.10) | −1.5 | (2.08) | 0.2 | (1.76) | −1.8 | (4.00) |
| Day 28: n | 22 | | 22 | | 24 | | 7 | | 7 | | 8 | |
| Mean (SD) | −3.8 | (3.31) | −4.5 | (3.58) | −7.1 | (4.63) | 0.2 | (3.63) | −0.7 | (4.06) | −0.7 | (3.81) |
| Day 42: n | 23 | | 22 | | 24 | | 7 | | 7 | | 8 | |
| Mean (SD) | −3.5 | (4.51) | −5.3 | (3.65) | −8.0 | (4.98) | −1.6 | (4.93) | 1.9 | (3.61) | −1.1 | (5.31) |

1 or 2 g versus 4 g

| | | | | | | |
|---|---|---|---|---|---|---|
| LS Mean | 1.5 | 1.5 | — | — | — | — |
| CI | 0.2, 2.9 | 0.1, 2.9 | — | — | — | — |
| p-value | 0.027 | 0.032 | | | | |

TABLE 6
EFA Parameter EPA/AA (Plasma and RBC) Mean change from Baseline to Days 14, 28 and 42.

| EFA | Ethyl-EPA | | | Placebo | | |
|---|---|---|---|---|---|---|
| Parameter | 1 g | 2 g | 4 g | 1 g | 2 g | 4 g |
| (μg/g) | (N = 23) | (N = 24) | (N = 24) | (N = 7) | (N = 8) | (N = 8) |

Plasma

| Baseline: n | 23 | 24 | 24 | 7 | 8 | | 8 |
|---|---|---|---|---|---|---|---|
| Mean (SD) | 0.2 (0.14) | 0.2 (0.12) | 0.2 (0.07) | 0.2 (0.11) | 0.2 | (0.10) | 0.2 (0.07) |
| Day 14: n | 23 | 23 | 24 | 7 | 7 | | 8 |
| Mean (SD) | 0.3 (0.4) | 0.6 (0.23) | 1.1 (0.28) | 0.0 (0.09) | 0.0 | (0.12) | 0.1 (0.12) |
| Day 28: n | 22 | 22 | 24 | 7 | 7 | | 8 |
| Mean (SD) | 0.3 (0.20) | 0.8 (0.35) | 1.3 (0.42) | 0.0 (0.08) | 0.0 | (0.09) | 0.0 (0.06) |
| Day 42: n | 23 | 22 | 24 | 7 | 7 | | 8 |
| Mean (SD) | 0.3 (0.24) | 0.7 (0.29) | 1.3 (0.45) | 0.0 (0.10) | 0.0 | (0.12) | 0.0 (0.08) |

1 or 2 g versus 4 g

| | | | | | | |
|---|---|---|---|---|---|---|
| LS Mean | −0.66 | −0.41 | — | — | — | — |
| CI | −0.731, −0.597 | −0.475, −0.341 | — | — | — | — |
| p-value | <0.001 | <0.001 | | | | |

RBC

| Baseline: n | 23 | 24 | 24 | 7 | 8 | | 8 |
|---|---|---|---|---|---|---|---|
| Mean (SD) | 0.1 (0.07) | 0.1 (0.06) | 0.1 (0.04) | 0.1 (0.04) | 0.1 | (0.06) | 0.1 (0.03) |
| Day 14: n | 23 | 22 | 24 | 7 | 7 | | 8 |
| Mean (SD) | 0.1 (0.04) | 0.2 (0.04) | 0.3 (0.07) | 0.0 (0.03) | −0.0 | (0.05) | 0.0 (0.03) |
| Day 28: n | 22 | 22 | 24 | 7 | 7 | | 8 |
| Mean (SD) | 0.1 (0.05) | 0.02 (0.06) | 0.4 (0.11) | 0.0 (0.01) | −0.0 | (0.04) | 0.0 (0.02) |
| Day 42: n | 23 | 22 | 24 | 7 | 7 | | 8 |
| Mean (SD) | 0.1 (0.06) | 0.3 (0.06) | 0.4 (0.14) | 0.0 (0.03) | −0.0 | (0.05) | 0.0 (0.03) |

1 or 2 g versus 4 g

| | | | | | | |
|---|---|---|---|---|---|---|
| LS Mean | −0.18 | −0.11 | — | — | — | — |
| CI | −0.204, −0.162 | −0.126, −0.085 | — | — | — | — |
| p-value | <0.001 | <0.001 | | | | |

### Example 3. EPA Treatment of Hemodialysis Patients

The increased CV risk seen in patients on hemodialysis does not appear to be driven by dyslipidemia and other traditional risk factors involved in the general population. Indeed, interventions that target these traditional risk factors, such as statins, antihypertensive drugs, and weight loss do not lower CV risk in this population even though they are effective at lowering CV risk in other populations. Instead, oxidative stress and chronic inflammation appear to be important drivers of CVD in the hemodialysis population.

Four grams per day of icosapent ethyl (E-EPA) are administered to patients on hemodialysis, compared to administration of placebo. E-EPA therapy corrects the EPA deficiency in the hemodialysis patients, increases the EPA/AA ratio associated with adverse CV events and markers of inflammation in this population, and reduces cardiovascular disease risk compared to subjects receiving placebo.

### Example 4. EPA Treatment of Patients Having Diabetes and/or Cardiovascular Disease on Concomitant or Stable Statin Therapy without End-Stage Renal Disease

Low estimated glomerular filtration rate (eGFR) is associated with dyslipidemia and other CV risk factors, and is an independent predictor of CV mortality. Real-world renal function data in high CV risk statin-treated patients with elevated triglycerides (TG) was analyzed using a retrospective administrative claims study (Optum Research Database).

The patients that were analyzed are at least about 45 years of age, have diabetes and/or CV disease, such as atherosclerotic CV disease, do not have end-stage renal disease, are not receiving hemodialysis or peritoneal dialysis, and filled a statin prescription in 2010. A cohort of patients having high TG levels (e.g., TG≥150 mg/dL) and a propensity-matched comparator cohort having low TG levels (e.g., TG<150 mg/dL) and HDL-C levels greater than about 40 mg/dL were assessed and followed for at least about 6 months or less due to death. Patients in each cohort were administered four (4) grams per day of icosapent ethyl (E-EPA)

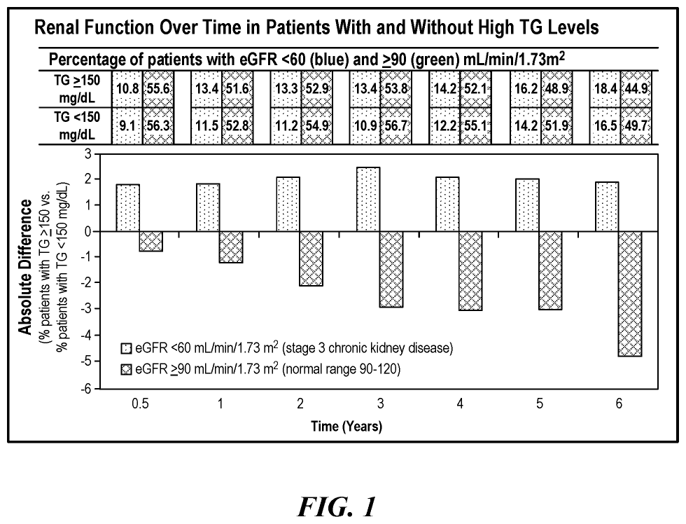

Comparing the high-TG cohort to the comparator cohort (both n=23,181), mean (SD) baseline eGFR was 90.1 (20.2) vs 90.7 (18.9) mL/min/1.73 m² and mean (SD) follow-up was 41.4 (23.7) vs 42.5 (23.9) months. During more than five (5) years of follow-up, the percentage of patients having an eGFR level of less than about 60 increased in both, but the percentage was higher in the high-TG cohort at each time point. Conversely, the percentage of patients having an eGFR level of at least about 90 decreased in both, but was lower in the high-TG cohort at each time point (FIG. 1). These differences between the two cohorts were not significant for the percentage of patients in each eGFR category but these differences widened over time.

This real-world analysis of statin-treated patients having a high CV risk suggests a trend toward greater worsening of renal function (e.g., long-term renal function) in patients with high TG compared to patients without high TG over time.

### Example 5. EPA Treatment of Patients Having Reduced Kidney Function and Diabetes Mellitus Reduces One or More CVD-Related Parameters

CVD-related (e.g., atherogenic) parameters were analyzed in a post-hoc study of 198 patients from the multi-center, placebo-controlled, randomized double-blind, 12-week study described in Example 1. These 198 patients have reduced kidney reduced function (e.g., eGFR<90 mL/min/1.73 m² for ≥3 months) and diabetes mellitus.

Objective. The primary objective of this post-hoc study was to determine if and how 4 g of daily of >96% E-EPA, compared to placebo, affects patients with reduced kidney function, diabetes mellitus, and high triglyceride levels. The secondary objective was to determine if and how 4 g daily >96% E-EPA, compared to placebo, affected CVD-related parameters in patients having a high risk for CVD and fasting TG levels □200 mg/dL and <500 mg/dL, despite treatment to LDL-C goal on statin therapy. These CVD-parameters include lipid levels, lipoprotein levels, and inflammatory markers, such as TC, LDL-C, HDL-C, VLDL-C, VLDL-TG, RLP-C, non-HDL-C, Apo B, Apo C-III, Lp-PLA₂, hsCRP, and ox-LDL, and in plasma and RBC EPA concentration. CVD-parameters were analyzed for median difference in percent change from baseline to week 12 in subjects receiving 4 g/day of E-EPA and those receiving the placebo.

Subjects: 98 patients were randomized to E-EPA and 100 patients were randomized to placebo. In total, atherogenic parameters were analyzed in all 198 patients, 56% of which were male and 97% of which were white. The mean age of the 198 patients was 63 years old. Patients were excluded from the study if they had a known nephrotic range (>3 g/day) proteinuria, history or evidence of major and clinically significant renal disease that would interfere with the conduct of the study or interpretation of the data, or required peritoneal dialysis or hemodialysis for renal insufficiency. The baseline characteristics in patients randomized to E-EPA and placebo were similar as shown in Table 7.

TABLE 7
Baseline Characteristics of Patients with Diabetes Mellitus
and eGFR <90 mL/min/1.73 m² for ≥3 months.

| | Icosapent Ethyl | | Placebo | |
|---|---|---|---|---|
| Parameter | 4 g/day (n = 98) | | (n = 100) | |
| Age, mean (SD), years | 63 | (8.8) | 63 | (9.6) |
| Males, n (%) | 56 | (57) | 54 | (54) |
| White, n (%) | 95 | (97) | 97 | (97) |
| Body mass index, mean (SD), kg/m² | 32.9 | (4.9) | 33.9 | (4.9) |
| eGFR, median (IQR), mL/min/1.73 | 76.4 | (15.2) | 74.9 | (20.8) |
| m² | | | | |

In this study, 6 patients in the placebo group discontinued (i.e., withdrawal with consent [n=2]; adverse event [n=2]; death [n=1]; other reason [n=1] and 4 patients from the 4 g/day group discontinued (i.e., adverse event [n=3] and other reason [n=1]). The death in the placebo group was due to myocardial infarction and not considered to be assigned to the investigational agent.

Analysis: The median difference in lipid/lipoprotein, apolipoprotein, and inflammatory and oxidation parameters were determined from baseline to week 12 for 4/g day of E-EPA compared to placebo. The primary efficacy variable was median difference in percent change in triglyceride levels from baseline to week 12.

Results: Tables 8 and 9 provide the effects of 4 g/day of E-EPA on patient's lipid/lipoprotein, apolipoprotein, and markers of oxidation and inflammation. As demonstrated in Tables 8 and 9, 4 g/day of E-EPA decreased triglyceride levels and also significantly improved apolipoproteins and markers of oxidation and inflammation.

TABLE 8
Effects of E-EPA on Lipids/Lipoproteins in Patients with Diabetes Mellitus and eGFR <90 mL/min/1.73 m² for ≥3 months.

| | | | | | | | | | | | | | Median Difference |
|---|---|---|---|---|---|---|---|---|---|---|---|---|---|
| | | | | | | | | | | | | | in % Change From |
| | Icosapent Ethyl 4 g/day (n = 98) | | | | | | Placebo (n = 100) | | | | | | Baseline for Icosapent |
| | | | End of | | Change From | | | | End of | | Change From | | Ethyl 4 g/day vs. |
| Lipids/Lipoproteins | Baseline | | Treatment | | Baseline, % | | Baseline | | Treatment | | Baseline, % | | Placebo, %, P |
| TG (mg/dL) (Primary | 262.5 | (90.0) | 209.8 | (83.5) | −19.3 | (30.9) | 259.0 | (73.3) | 266.8 | (127.0) | 1.6 | (35.5) | −19.7, <0.0001 |
| efficacy variable) | | | | | | | | | | | | | |
| LDL-C (mg/dL) | 82.5 | (29.0) | 83.0 | (29.0) | −2.1 | (28.2) | 81.5 | (29.5) | 83.5 | (32.5) | 10.0 | (34.9) | −7.1, 0.0375 |
| Non-HDL-C (mg/dL) | 128.5 | (34.0) | 120.5 | (39.0) | −6.0 | (21.2) | 128.0 | (36.0) | 134.5 | (41.5) | 8.7 | (23.4) | −13.7, <0.0001 |
| TC (mg/dL) | 169.5 | (40.0) | 160.5 | (38.0) | −5.2 | (16.5) | 168.0 | (41.5) | 181.0 | (44.0) | 8.6 | (20.2) | −12.5, <0.0001 |
| HDL-C (mg/dL) | 36.0 | (12.0) | 37.0 | (10.0) | −3.0 | (18.3) | 38.5 | (12.5) | 40.0 | (14.0) | 6.7 | (21.6) | −7.0, 0.0009 |
| VLDL-C (mg/dL) | 42.0 | (20.0) | 36.5 | (23.0) | −12.3 | (45.6) | 44.5 | (18.0) | 47.0 | (28.0) | 5.5 | (58.6) | −19.0, 0.0006 |
| VLDL-TG (mg/dL) | 186.0 | (84.0) | 146.0 | (76.0) | −19.2 | (43.0) | 185.5 | (81.0) | 194.0 | (114.5) | 6.4 | (51.1) | −22.3, <0.0001 |
| RLP-C (mg/DL)* | 14.0 | (7.0) | 9.5 | (5.0) | −28.9 | (45.0) | 15.0 | (6.0) | 16.0 | (12.0) | 13.0 | (64.2) | −35.4, 0.0029 |

Data are presented as median (IQR) for endpoint values.
Median differences in percent changes from baseline versus placebo are Hodges-Lehmann medians.
*n = 34 for Icosapent ethyl 4 g/day, n = 33 for placebo; RLP-C was only measured in approximately the first 35% of patients randomized in the ANCHOR study.

TABLE 9
Effects of E-EPA on Apolipoproteins and Markers of Oxidation and Inflammation
in Patients with Diabetes Mellitus and eGFR <90 mL/min/1.73 m² for ≥3 months.

| | | | | | | | Median Difference |
|---|---|---|---|---|---|---|---|
| | | | | | | | in % Change From |
| | Icosapent Ethyl 4 g/day (n = 98) | | | Placebo (n = 100) | | | Baseline for Icosapent |
| | | End of | Change From | | End of | Change From | Ethyl 4 g/day vs. |
| Parameter | Baseline | Treatment | Baseline, % | Baseline | Treatment | Baseline, % | Placebo, %, P |

Apolipopproteins

| | | | | | | | |
|---|---|---|---|---|---|---|---|
| Apo B (mg/dL)* | 92.0 (22.0) | 88.0 (25.0) | −3.5 (17.0) | 91.0 (24.0) | 93.0 (23.0) | 7.3 (23.5) | −10.1, <0.0001 |
| n = 95, 97 | | | | | | | |
| Apo C-III (mg/dL)* | 42.0 (20.0) | 36.5 (23.0) | −12.3 (45.6) | 44.5 (18.0) | 47.0 (28.0) | 5.5 (58.6) | −19.0, 0.0006 |
| n = 98, 100 | | | | | | | |

Markers of Oxidation and Inflammation

| | | | | | | | |
|---|---|---|---|---|---|---|---|
| Ox-LDL (U/L)† | 54.2 (15.8) | 49.5 (12.8) | −8.2 (17.8) | 52.1 (17.5) | 59.4 (23.2) | 13.1 (29.6) | −16.2, <0.0001 |
| n = 41, 43 | | | | | | | |
| Lp-PLA₂ (ng/mL) | 178.0 (54.0) | 160.0 (52.0) | −14.4 (16.5) | 187.0 (61.0) | 197.0 (66.0) | 4.0 (23.9) | −16.6, <0.0001 |
| n = 95, 95 | | | | | | | |
| hsCRP (mg/L) | 2.2 (2.9) | 1.7 (3.0) | −2.4 (81.7) | 2.6 (3.9) | 2.8 (4.5) | 20.0 (92.8) | −24.4, 0.0120 |
| n = 95, 97 | | | | | | | |

Data are presented as median (IQR) for endpoint values.
Median differences in percent changes from baseline versus placebo are Hodges-Lehmann medians.
Patient numbers are presented as Icosapent ethyl 4 g/day and placebo, respectively.
*Apo B and Apo C-III levels were measured in the subgroup of all patients with available archived plasma samples from the ANCHOR study.²
†Ox-LDL was only measured in approximately the first 35% of patients randomized in the ANCHOR study.

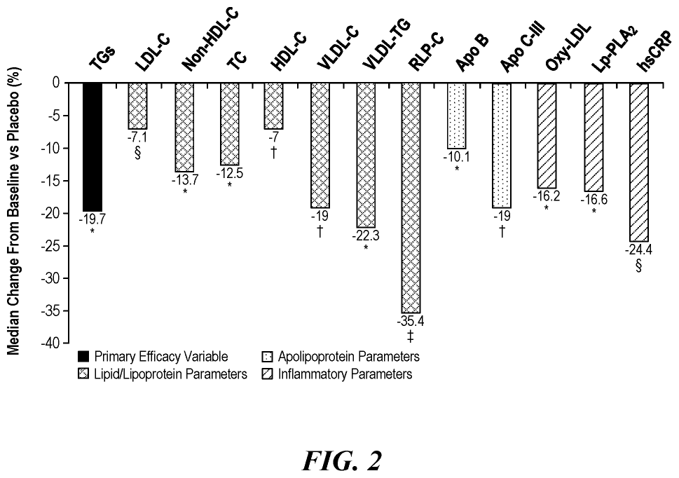

These results are further exemplified in FIG. 2 which shows that 4 g/day of E-EPA significantly reduced triglycerides by about 19.7% (P<0.0001), without increasing LDL-C levels, and significantly improved other atherogenic and inflammatory parameters in patients with reduced kidney function (e.g., eGFR<90 mL/min/1.73 m² for ≥3 months) and diabetes mellitus compared to placebo. Patients receiving 4 g/day of E-EPA after 12 weeks experienced no significant change in serum creatinine (e.g., 0.9 mg/dL to 0.9 mg/dL), albumin (e.g., 4.5 g/dL to 4.5 g/dL) levels, eGFR levels (e.g., 75.4 to 79.1 mL/min/1.73 m²), and BUN (e.g., 18 mg/dL to 18 mg/dL) before and after the 12 weeks. In addition, EPA levels increased 676.2% in plasma and 611.7% in RBC in patients who received E-EPA compared to those who received placebo (both P>0.0001) as shown in Table 10.

TABLE 10
Change in EPA Levels in Plasma and RBCs from Baseline to Week 12 in Patients
with Diabetes Mellitus and eGFR <90 mL/min/1.73 m² for ≥3 months.

| | | | | | | | Change From |
|---|---|---|---|---|---|---|---|
| | Icosapent Ethyl 4 g/day (n = 98) | | | Placebo (n = 100) | | | Baseline, LS |
| | | End of | Change From | | End of | Change From | Mean, Icosapent |
| | Baseline, | Treatment, | Baseline, LS | Baseline, | Treatment, | Baseline, LS | Ethyl 4 g/day vs. |
| Parameter | Mean (SD) | Mean (SD) | Mean (SE) | Mean (SD) | Mean (SD) | Mean (SE) | Placebo, %, P |
| Plasma EPA, μg/mL* | 24.6 (9.0) | 179.7 (62.4) | 157.5 (10.1) | 31.2 (37.0) | 33.6 (37.4) | 4.5 (9.7) | 676.2, <0.0001 |
| n = 36, 40 | | | | | | | |
| RBC EPA, μg/mL* | 11.4 (5.0) | 71.9 (23.7) | 62.1 (2.7) | 12.1 (7.3) | 10.3 (6.7) | −0.2 (2.6) | 611.7, <0.0001 |
| n = 36, 41 | | | | | | | |

*EPA levels were measured in approximately the first 216 patients with complete sample datasets as prespecified by the study protocol.
Patient numbers are presented as Icosapent ethyl 4 g/day and placebo, respectively.

Overall, the safety profile for patients randomized to 4 g/day E-EPA was similar to patients randomized to placebo as shown in Table 11.

TABLE 11
Adverse Events in Patients with Diabetes Mellitus and
eGFR <90 mL/min/1.73 m² for ≥3 months.

Patients With TEAEs, n (%)

| | Icosapent Ethyl | Placebo |
|---|---|---|
| Preferred Term | 4 g/day (n = 100) | (n = 102) |
| Any TEAE | 49 (49.0) | 53 (52.0) |
| Nausea* | 3 (3.0) | 3 (2.9) |
| Diarrhea* | 5 (5.0) | 6 (5.9) |
| Nasopharyngitis* | 1 (1.0) | 1 (1.0) |
| Arthralgia*† | 1 (1.0) | 0 |

*TEAS occurring in >3% of all patients in the entire safety population of the ANCHOR study.⁷
†Most commonly reported AE listed in the Icosapent ethyl prescribing information.⁵

Conclusion. In statin-treated patients with persistently high triglyceride levels of about 200 mg/dL to about 499 mg/dL, diabetes mellitus, and reduced kidney function (i.e., eGFR<90 mL/min/1.73 m² for ≥3 months), 4 g/day E-EPA significantly reduced triglyceride levels and other atherogenic and inflammatory parameters without increase LDL-C vs placebo. 4 g/day of E-EPA was well tolerated with a safety profile similar to that of the placebo and with no negative impact on renal function.

### Example 6. EPA Treatment of Patients Having Reduced Kidney Function and HS-CRP Levels ≥2.0 mg/L Reduces One or More CVD-Related Parameters

CVD-related (e.g., atherogenic) parameters were analyzed in a post-hoc study of 152 patients from the multi-center, placebo-controlled, randomized double-blind, 12-week study described in Example 1. These 152 patients have reduced kidney reduced function (e.g., eGFR<90 mL/min/1.73 m² for ≥3 months) and hsCRP≥2.0 mg/L.

Subjects: 72 patients were randomized to E-EPA and 80 patients were randomized to placebo. In total, atherogenic parameters were analyzed in all 152 patients, 54% of which were male and 99% of which were white. The mean age of the 152 patients was 64 years old.

The primary objective of this post-hoc study, was to determine if and how 4 g of daily of >96% E-EPA, compared to placebo, affects patients with reduced kidney function, elevated hsCRP (e.g, hsCRP≥2.0 mg/L), and high triglyceride levels. The secondary objective was to determine if and how 4 g daily of >96% E-EPA, compared to placebo, affected CVD-related parameters in patients with a high risk for CVD and fasting TG levels □200 mg/dL and <500 mg/dL, despite treatment to LDL-C goal on statin therapy. These CVD-parameters include lipid levels, lipoprotein levels, and inflammatory markers, such as TC, LDL-C, HDL-C, VLDL-C, VLDL-TG, RLP-C, non-HDL-C, Apo B, Apo C-III, Lp-PLA, hsCRP, and ox-LDL, and in plasma and RBC EPA concentration. CVD-parameters were analyzed for median difference in percent change from baseline to week 12 in subjects receiving 4 g/day of E-EPA and those receiving the placebo.

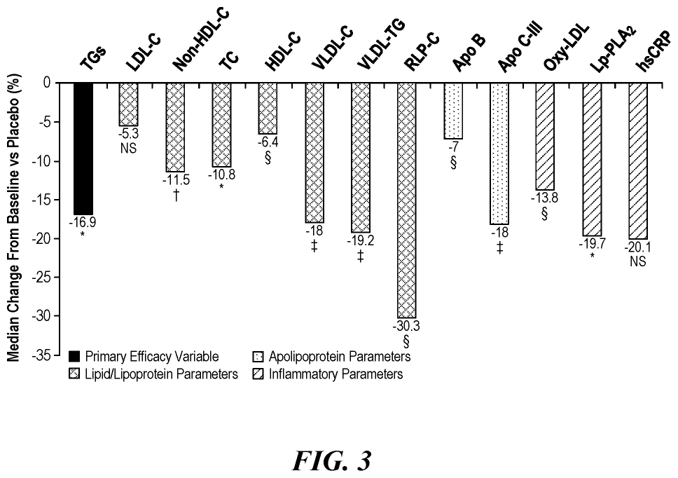

Analysis: The median difference in lipid/lipoprotein, apolipoprotein, and inflammatory parameters were determined from baseline to week 12 for 4/g day of E-EPA compared to placebo. These results are shown in FIG. 3.

Results: As shown in FIG. 3, 4 g/day of E-EPA significantly reduced triglycerides by about 16.9% (P<0.0001), without increasing LDL-C levels, and significantly improved other atherogenic and inflammatory parameters in patients with reduced kidney function (e.g., eGFR<90 mL/min/1.73 m² for ≥3 months) and diabetes mellitus compared to placebo. Patients receiving 4 g/day of E-EPA after 12 weeks experienced no change in serum creatinine (e.g., 0.9 mg/dL to 0.9 mg/dL) levels and no statistically significant change albumin (e.g., 4.5 g/dL to 4.4 g/dL) levels and an increase of eGFR from 72.4 to 78.5 mL/min/1.73 m² before and after the 12 weeks. In addition, EPA levels increased 662.7% in plasma and 622.0% in RBC in patients who received E-EPA compared to those who received placebo (both P>0.0001).

### Example 7. EPA Treatment of Patients Having Chronic Kidney Disease Reduces One or More CVD-Related Parameters In

CVD-related (e.g., atherogenic) parameters were analyzed in a post-hoc study of 55 patients from the multi-center, placebo-controlled, randomized double-blind, 12-week study described in Example 1. These 55 patients have chronic kidney disease (e.g., eGFR<60 mL/min/1.73 m² for ≥3 months). There was no lower limit of eGFR specified.

Subjects: 19 patients were randomized to E-EPA and 36 patients were randomized to placebo. One patient in each group discontinued and was excluded from efficacy analyses (but not safety analyses). EPA levels in plasma and RBC were measured in 23 of the 55 patients. These measurements were taken on 7 of the patients randomized to E-EPA and on 16 of the patients that were randomized to the placebo. Table 12 shows that the baseline characteristics of the placebo and 4/day of E-EPA treatment groups were similar and that these patients had a mean eGFR baseline of 50.4 mL/min/1.73 m² for ≥3 months with a range of 24.7 to 59.7 mL/min/1.73 m² for ≥3 months.

The primary objective of this post-hoc study, which is based on an intention to treat, was to determine if and how 4 g daily of >96% E-EPA affects patients with stage 3 chronic kidney disease (e.g., eGFR<60 mL/min/1.73 m² for ≥3 months). The secondary objective was to determine if and how 4 g daily of >96% E-EPA, compared to a placebo, affected CVD-related parameters in patients with a high risk of CVD and fasting TG levels ≥200 mg/L and <500 mg/dL, despite LDL-C goal on statin therapy. These CVD-parameters include lipid levels, lipoprotein levels, and inflammatory markers, such as TC, LDL-C, HDL-C, VLDL-C, VLDL-TG, RLP-C, non-HDL-C, Apo B, Apo C-III, Lp-PLA, hsCRP, and ox-LDL, and in plasma and RBC EPA concentration. CVD-parameters were analyzed for median difference in percent change from baseline to week 12 in subjects receiving 4 g/day of E-EPA and those receiving the placebo.

Analysis: The median difference in percent change from baseline between 4 g/day of E-EPA and placebo for the primary efficacy variable and additional assessments were estimated using the Hodges-Lehmann method (P values from the Wilcoxon rank-sum test for treatment comparisons). Using the Hodges-Lehmann method, departures from normal distribution were observed; for normally distributed parameters, an analysis of covariance model was used with least squares (LS) means and standard errors (SE). Missing values were imputed using the last-observation-carried-forward method. For all post hoc analyses, the alpha for statistical significance was 0.05.

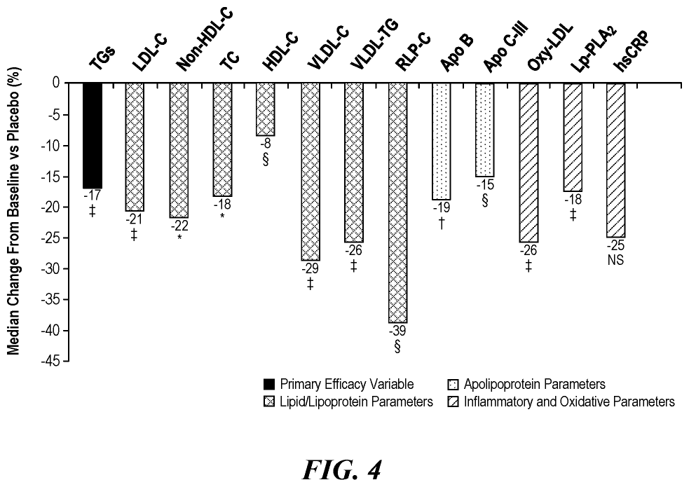

Results: As shown in Table 13 and FIG. 4, 4 g/day of E-EPA significantly reduced TG by at least about 16.9% (P=0.007) in patients with chronic kidney disease (e.g., eGFR<60 mL/min/1.73 m² for ≥3 months) compared to placebo. 4 g/day of E-EPA also significantly reduced other atherogenic and inflammatory parameters to include significantly reduced LDL-C, non-HDL-C, TC, HDL-C, VLDL-C, VLDL-TGs, and RLP-C compared with placebo (P<0.05 to <0.0001), as well as both Apo B and Apo C-III (P=0.0002 and P=0.0168, respectively) compared to placebo control. 4 g/day of E-EPA also significantly reduced marker of oxidation and inflammation to include ox-LDL and Lp-PLA₂ (both P=0.0068). There were no statistically significant changes hsCRP (P=0.1209) with 4 g/day of E-EPA compared to a placebo control. 4 g/day of E-EPA significantly reduced median TGs from baseline by −16.9% in the CKD cohort compared with −21.5% in the entire ANCHOR population.

Plasma and RBC Levels of EPA: 4 g/day of E-EPA significantly increased mean (standard deviation [SD]) plasma EPA levels from 22.8 (7.0) μg/mL at baseline to 233.4 (82.1) μg/mL at week 12; 4 g/day of E-EPA increased LS mean (SE) by 879.2% (75.8%) compared with placebo control (P<0.0001). Similarly, 4 g/day of E-EPA significantly increased mean (SD) EPA levels in RBC from 10.6 (2.9) μg/mL at baseline to 72.3 (34.0) μg/mL at week 12; 4 g/day of E-EPA increased LS mean (SE) increase by 579.4% (73.0%) with compared with placebo control (P<0.0001).

4 g/day of E-EPA did not produce significant changes in serum creatinine, blood urea nitrogen (BUN), or eGFR from baseline to week 12 among patients with CKD compared to placebo control. The median (interquartile range [IQR]) serum creatinine in the 4 g/day E- of EPA group was 1.2 (0.3) mg/dL at baseline and 1.2 (0.4) mg/dL at week 12, and 1.3 (0.3) mg/dL at baseline and 1.1 (0.3) mg/dL at week 12 for the placebo control group. The median difference in change from baseline compared with placebo group was 6.5% (P=0.1983). Median (IQR) BUN was 24.5 (12.0) mg/dL at baseline and 22.5 (11.0) mg/dL at week 12 in the 4 g/day of E-EPA group, and 23.0 (7.0) mg/dL at baseline and 23.0 (11.0) mg/dL at week 12 in the placebo group. The median difference in change from baseline compared with placebo group was 1.0 mg/dL (P=0.5471) or 5.5% (P=0.5291). Median (IQR) eGFR was 55.0 (13.8) mL/min/1.73 m² at baseline and 56.0 (12.9) mL/min/1.73 m² at week 12 in the 4 g/day of E-EPA group, and 52.4 (11.2) mL/min/1.73 m² at baseline and 58.8 (14.5) mL/min/1.73 m² at week 12 in the placebo group. The median difference in change from baseline compared with placebo was −7.8% (P=0.1983). Baseline and end-of-treatment levels of serum albumin were similar in both the 4 g/day group and placebo group, and there were no significant median changes from baseline in either group over the 12-week study.

Safety analysis: TEAE were reported in 11 of 19 (57.9%) patients with CKD in the 4 g/day of E-EPA group and 20 of 36 (55.6%) patients in the placebo group. Among TEAEs reported most frequently in the overall ANCHOR population (n=702), diarrhea was reported by 2 patients with CKD (i.e, 10.5%) in the 4 g/day of E-EPA group and 2 patients with CKD (i.e., 5.6%) in the placebo group. Nausea, nasopharyngitis, and arthralgia were not reported by any patients with CKD in the 4 g/day of E-EPA or placebo groups.

As with the overall ANCHOR population of Example 1 above, the safety profiles of 4/g day of E-EPA were similar to placebo in this subgroup analysis of patients with CKD (e.g., eGFR<60 mL/min/1.73 m² for ≥3 months).

TABLE 12
Baseline Characteristics of Patients from ANCHOR study Having
CKD (e.g., eGFR <60 mL/min/1.73 m² for ≥3 months)

| | Icosapent Ethyl | | Placebo | |
|---|---|---|---|---|
| Variable | 4 g/day (n = 18 | | (n = 35) | |
| Age, mean (SD), years | 68.2 | (7.2) | 68.0 | (8.4) |
| Sex, n (%) | | | | |
| Male | 10 | (55.6) | 17 | (48.6) |
| Female | 8 | (44.4) | 18 | (51.4) |
| Race, n (%) | | | | |
| White | 18 | (100) | 34 | (97.1) |
| Body mass index, mean (SD), kg/m² | 32.2 | (4.8) | 33.4 | (5.5) |
| eGFR, mean (SD), mL/min/1.73 m² | 50.5 | (10.6) | 50.3 | (7.3) |
| Diabetes, n (%) | 10 | (55.6) | 24 | (68.6) |

CKD, chronic kidney disease; eGFR, estimated glomerular filtration rate; ITT, intent-to-treat; SD, standard deviation.

TABLE 13
Effects of Icosapent Ethyl 4 g/day on Lipid and Lipoprotein Parameters and on Markers of Oxidation
and Inflammation from the ANCHOR Study with CKD (e.g., eGFR <60 mL/min/1.73 m² for ≥3 months)

| | | | | | | | Median Difference |
|---|---|---|---|---|---|---|---|
| | | | | | | | in % Change From |
| | Icosapent Ethyl 4 g/day | | | Placebo | | | Baseline, Icosapent |
| | | End of | Change From | | End of | Change From | Ethyl 4 g/day vs. |
| Parameter | Baseline | Treatment | Baseline, % | Baseline | Treatment | Baseline, % | Placebo, %, P |

Lipid Parameters

| | | | | | | | | | | | | | |
|---|---|---|---|---|---|---|---|---|---|---|---|---|---|
| TGs (mg/dL) | 268.5 | (101.5) | 230.5 | (66.0) | −17.1 | (43.2) | 262.0 | (70.5) | 272.5 | (98.5) | 5.9 | (36.0) | −16.9, 0.0074 |
| (primary efficacy variable) | | | | | | | | | | | | | |
| n = 18, 35 | | | | | | | | | | | | | |
| LDL-C (mg/dL) | 76.0 | (24.0) | 70.5 | (24.0) | −6.5 | (19.9) | 74.0 | (38.0) | 84.0 | (27.0) | 13.7 | (38.9) | −20.8, 0.0086 |
| n = 18, 35 | | | | | | | | | | | | | |
| Non-HDL-C (mg/dL) | 124.5 | (34.0) | 116.0 | (21.0) | −8.4 | (7.7) | 131.0 | (31.0) | 141.0 | (45.0) | 10.7 | (34.3) | −21.5, <0.0001 |
| n = 18, 35 | | | | | | | | | | | | | |
| TC (mg/dL) | 159.5 | (49.0) | 151.5 | (41.0) | −8.1 | (8.2) | 165.0 | (43.0) | 176.0 | (40.0) | 9.5 | (21.0) | −18.2, <0.0001 |
| n = 18, 35 | | | | | | | | | | | | | |
| HDL-C (mg/dL) | 35.0 | (13.0) | 35.5 | (14.0) | 0.9 | (12.4) | 40.0 | (15.0) | 40.0 | (21.0) | 10.4 | (25.0) | −8.3, 0.0362 |
| n = 18, 35 | | | | | | | | | | | | | |
| VLDL-C (mg/dL) | 42.0 | (12.0) | 36.5 | (12.0) | −16.3 | (40.4) | 47.0 | (19.0) | 55.0 | (20.0) | 10.0 | (46.0) | −28.7, 0.0026 |
| n = 18, 35 | | | | | | | | | | | | | |
| VLDL-TGs (mg/dL) | 194.0 | (102.0) | 168.5 | (65.0) | −25.8 | (56.6) | 185.0 | (89.0) | 209.0 | (115.0) | 0.0 | (59.7) | −25.6, 0.0020 |
| n = 18, 35 | | | | | | | | | | | | | |
| RLP-C (mg/dL)* | 17.0 | (7.0) | 10.0 | (2.0) | −38.9 | (40.6) | 14.5 | (7.0) | 14.0 | (6.0) | 6.3 | (62.4) | −38.8, 0.0295 |
| n = 9, 14 | | | | | | | | | | | | | |

Lipoprotein Parameters

| | | | | | | | | | | | | | |
|---|---|---|---|---|---|---|---|---|---|---|---|---|---|
| Apo B (mg/dL)† | 87.0 | (27.0) | 83.0 | (22.0) | −7.2 | (10.2) | 90.0 | (22.0) | 94.0 | (24.0) | 13.0 | (23.3) | −18.7, 0.0002 |
| n = 17, 35 | | | | | | | | | | | | | |
| Apo C-III (mg/dL)† | 15.6 | (3.9) | 13.9 | (2.6) | −7.3 | (24.4) | 16.2 | (3.2) | 17.0 | (4.0) | 10.0 | (23.1) | −15.0, 0.0168 |
| n = 13, 32 | | | | | | | | | | | | | |

Markers of Oxidation and Inflammation

| | | | | | | | | | | | | | |
|---|---|---|---|---|---|---|---|---|---|---|---|---|---|
| Ox-LDL (u/L)* | 48.6 | (19.7) | 45.1 | (21.6) | −9.5 | (8.6) | 53.7 | (10.5) | 55.5 | (21.6) | 16.7 | (31.6) | −25.7, 0.0068 |
| n = 8, 17 | | | | | | | | | | | | | |
| Lp-PLA₂ (ng/mL) | 173.0 | (65.0) | 150.0 | (68.0) | −12.7 | (21.2) | 197.0 | (68.0) | 202.5 | (74.0) | 7.0 | (21.5) | −17.5, 0.0068 |
| n = 17, 34 | | | | | | | | | | | | | |
| hsCRP (mg/L) | 1.9 | (1.8) | 2.1 | (2.9) | −16.9 | (26.1) | 2.6 | (3.8) | 2.6 | (6.3) | 9.1 | (103.3) | −25.0, 0.1209 |
| n = 17, 35 | | | | | | | | | | | | | |

Data are presented as median (interquartile range) for endpoint values.
Median percent changes versus placebo are Hodges-Lehmann estimates.
Patient numbers are presented as icosapent ethyl 4 g/day and placebo, respectively.
*RLP-C and ox-LDL were only measured in approximately the first 35% of patients randomized in ANCHOR.
†Apo B and Apo C-III levels were measured in the subset of all patients with available archived plasma samples from ANCHOR.

What is claimed is:

1. A method of treating or preventing cardiovascular-related disease in a subject having baseline fasting triglycerides of about 200 mg/dl to about 500 mg/dl, reduced kidney function, and diabetes mellitus, the method comprising administering to the subject a composition comprising about 4 g of at least 96% ethyl eicosapentaenoate (E-EPA), by weight, of total fatty acids present in the composition per day for 12 weeks, wherein after administration of the composition, the subject experiences a reduction in triglycerides and cardiovascular risk factors without raising low density lipoprotein cholesterol (LDL-C) levels compared to a placebo control.

2. The method of claim 1, wherein the subject has an estimated glomerular filtration rate (GFR) levels less than 90 mL/min/1.73 m² for 3 months or greater.

3. The method of claim 1, wherein the subject has one or more of: (a) a fasting baseline high-sensitivity C-reactive protein (hs-CRP) level of at least about 2 mg/L, (b) a fasting baseline lipoprotein-associated phospholipase A2 (Lp-PLA₂) level of 170 ng/dl; (c) a fasting oxidized low density lipoprotein (ox-LDL) level of at least about 55 mg/dl; (d) a fasting baseline apolipoprotein C-III (Apo C-III) level of at least about 40 mg/dl; (e) a fasting baseline apolipoprotein B (Apo B) level of at least about 100 mg/dl; (f) a fasting baseline level of remnant-like particles cholesterol (RLP-C) level of at least about 40 mg/dl; (g) a fasting baseline level of very low density lipoprotein triglyceride (vLDL-TG) level of at least about 180 mg/dl; (h) a fasting baseline high density lipoprotein cholesterol (HDL-C) level of not more than about 40 mg/dl; (i) a fasting baseline very low density lipoprotein (vLDL-C) level of at least about 40 mg/dl; (j) a fasting baseline total cholesterol (TC) level of greater than about 160 mg/dl; (k) a baseline fasting non-high density lipoprotein cholesterol (non-HDL-C) level of at least about 120 mg/dl; (l) a fasting baseline albumin level at least about 4.5 mg/dl; (m) a fasting baseline serum creatine level of at least about 0.9 mg/dl; or (n) a fasting baseline blood urea nitrogen (BUN) level of at least about 18 mg/dl.

4. The method of claim 1, wherein the subject exhibits a reduction in one or more of: hs-CRP; Lp-PLA₂; ox-LDL; Apo C III; Apo B; RLP-C; vLDL-TG; HDL-C; vLDL-C; TC; and non-HDL-C following administration of E-EPA as compared to placebo control and/or baseline.

5. The method of claim 3, wherein the subject exhibits one or more of: (a) a reduction in a hs-CRP level of at least about 25%, (b) a reduction in a Lp-PLA2 level of at least about 15%; (c) a reduction in an ox-LDL level of at least about 15%; (d) a reduction in an Apo C-III level of at least about 20%; (e) a reduction in an Apo B level of at least about 10%; (f) a reduction in a RLP-C level of at least about 35%; (g) a reduction in a vLDL-TG level of at least about 20%; (h) a reduction in a HDL-C level of at least about 7%; (i) a reduction in a vLDL-C level of at least about 15%; (j) a reduction in a TC level of at least about 10%; and (k) a reduction in a non-HDL-C level of at least about 10% as compared to baseline.

6. The method of claim 1, wherein the subject has an LDL-C level of about 40 mg/dl to about 100 mg/dl prior to said administering.

7. The method of claim 6, wherein the subject exhibits a reduction in LDL-C levels following administration of E-EPA as compared to placebo control and/or baseline.

8. The method of claim 1, wherein the subject has coronary heart disease or a coronary heart disease risk equivalent.

9. The method of claim 3, wherein the subject does not experience a statistically significant change in serum creatinine, eGFR levels, BUN and/or albumin levels following administration of E-EPA as compared to placebo control and/or baseline.

10. The method of 1, wherein the subject exhibits an increase in plasma and/or red blood cell (RBC) levels of E-EPA following administration of E-EPA as compared to placebo control and/or baseline.

11. The method of claim 1, wherein the subject is on concomitant statin therapy.

12. The method of claim 1, wherein the subject is on stable statin therapy.

13. The method of claim 1, wherein the subject has at least one cardiovascular-related disease.

14. The method of claim 1, wherein reduced kidney function is measured by the subject's GFR or the subject's estimated GFR.

15. The method of claim 1, wherein the risk factors are hs-CRP; Lp-PLA₂; ox-LDL; Apo C III; Apo B; RLP-C; vLDL-TG; HDL-C; vLDL-C; TC; and non-HDL-C.
